(19) United States
(12) Patent Application Publication
(10) Pub. No.: US 2025/0009352 A1
(43) Pub. Date: Jan. 9, 2025

(54) MOTORIZED SURGICAL HANDLE ASSEMBLY

(71) Applicant: Lexington Medical, Inc., Bedford, MA (US)

(72) Inventors: Andrew Marecki, West Boylston, MA (US); Leon Amariglio, Lexington, MA (US); David T. Moy, JR., Wellesley, MA (US); Richard Moyer, Allston, MA (US); Jameson Kief, Wakefield, MA (US)

(21) Appl. No.: 18/890,657

(22) Filed: Sep. 19, 2024

Related U.S. Application Data
(63) Continuation-in-part of application No. 18/496,107, filed on Oct. 27, 2023.
Application No. 18/496,107 is a continuation-in-part of application No. 17/833,302, filed on Jun. 6, 2022.
(60) Provisional application No. 63/197,934, filed on Jun. 7, 2021.

(51) Int. Cl. A61B 17/072; A61B 17/00; A61B 90/00

(52) U.S. Cl. CPC A61B 17/07207; A61B 2017/00017; A61B 2017/00039; A61B 2017/00075; A61B 2017/00367; A61B 2090/067

(57) ABSTRACT

The present disclosure includes apparatuses for a motorized surgical handle assembly. An example apparatus includes a non-movable handle, a movable handle, a drive assembly, a drivetrain, a sensor, and a controller. The movable handle is movably coupled to the non-movable handle. The drivetrain is operably coupled to the movable handle and configured to move the drive assembly. The sensor is configured to obtain a measurement corresponding to at least one of a position or orientation of the movable handle. The controller is operably coupled to the drivetrain and the sensor, and configured to (i) determine, based at least in part on the measurement, that the movable handle has moved away from the non-movable handle by at least a threshold amount, and (ii) operate the drivetrain based at least in part on the determination to move the movable handle.

### CROSS-REFERENCE TO RELATED APPLICATION(S)

This application is a continuation-in-part application of U.S. patent application Ser. No. 18/496,107, filed Oct. 27, 2023, which is a continuation-in-part application of U.S. patent application Ser. No. 17/833,302, filed Jun. 6, 2022, which claims priority to U.S. Provisional Patent Application No. 63/197,934 filed Jun. 7, 2021, the contents of which are all hereby incorporated by reference herein for all purposes.

### TECHNICAL FIELD

The present disclosure relates generally to surgical handle assemblies and to motor driven surgical devices.

### BACKGROUND

A surgical handle assembly can be used in a number of surgical devices. One example includes use as a surgical stapler. A surgical stapler is a fastening device used to clamp tissue between opposing jaw structures to join tissue using surgical fasteners. Surgical staplers can include two elongated members used to clamp the tissue. One of the elongated members can include one or more reloadable cartridges and the other elongated member can include an anvil that can be used to form a staple when driven from the reloadable cartridge. A surgical stapler can receive one or more reloadable cartridges. An example of reloadable cartridges can include having rows of staples having a linear length. For example, a row of staples can have a linear length between 30 mm and 60 mm. A staple can be ejected by actuation of a movable handle member that is a part of the surgical handle assembly of the surgical stapler.

Some surgical staplers are equipped with an electric motor which can provide the power to clamp tissue, deliver staples, and provide power for other aspects of a surgical stapler.

### BRIEF DESCRIPTION OF THE DRAWINGS

FIG. 1 is a schematic diagram of an apparatus including a motorized surgical handle assembly and a reloadable cartridge assembly in accordance with a number of embodiments of the present disclosure.

FIG. 2 is a schematic diagram of a motorized surgical handle assembly in accordance with a number of embodiments of the present disclosure.

FIG. 3A is a schematic diagram of an embodiment of a movable handle in accordance with a number of embodiments of the present disclosure.

FIG. 3B is a schematic diagram of an exploded view of a movable handle in accordance with a number of embodiments of the present disclosure.

FIG. 4 is a schematic diagram of a first handle half in accordance with a number of embodiments of the present disclosure.

FIG. 5 is a schematic diagram of a second handle half in accordance with a number of embodiments of the present disclosure.

FIGS. 6A, 6B, and 6C are schematic diagrams of a mechanism for grasping and ungrasping a reloadable cartridge assembly in accordance with a number of embodiments of the present disclosure.

FIGS. 7A and 7B are schematic diagrams of a disengagement mechanism in accordance with a number of embodiments of the present disclosure.

FIGS. 8A and 8B are schematic diagrams of a locking mechanism for a movable handle in accordance with a number of embodiments of the present disclosure.

FIGS. 9A, 9B, 9C, and 9D are schematic diagrams of a reset mechanism for a movable handle in accordance with a number of embodiments of the present disclosure.

FIGS. 10A and 10B are schematic diagrams of a drivetrain in accordance with a number of embodiments of the present invention.

FIG. 11 is a schematic of a motorized surgical handle assembly in accordance with a number of embodiments of the present disclosure.

FIG. 12 is a schematic diagram of an exploded view of a motorized surgical handle assembly in accordance with a number of embodiments of the present disclosure.

FIG. 13 is a schematic diagram of an exploded view of a motorized surgical handle assembly in accordance with a number of embodiments of the present disclosure.

FIG. 14 is a schematic diagram of a part of a motorized surgical handle assembly in accordance with a number of embodiments of the present disclosure.

FIGS. 15A, 15B, 15C, and 15D are schematic diagrams of the bailout mechanism for a motorized surgical handle assembly in accordance with a number of embodiments of the present disclosure.

FIGS. 16A and 16B are schematic diagrams of a circuit board that could be used for a motorized surgical handle assembly in accordance with a number of embodiments of the present disclosure.

FIGS. 17A, 17B, and 17C are schematic diagrams of a selector cam that could be used for a motorized surgical handle assembly in accordance with a number of embodiments of the present disclosure.

FIGS. 18A, 18B, and 18C show an example of a clamp and unclamp mechanism for a motorized surgical handle assembly in accordance with a number of embodiments of the present disclosure.

FIGS. 19A, 19B, 19C, and 19D show an example of a selector cam in various settings for a motorized surgical handle assembly in accordance with a number of embodiments of the present disclosure.

FIGS. 20A, 20B, and 20C show an example of a selector cam in a reverse position in accordance with a number of embodiments of the present disclosure.

FIGS. 21A and 21B show an example of a sensor usable with a movable handle for a motorized surgical handle assembly configured in accordance with a number of embodiments of the present disclosure.

FIG. 22 shows an example of a sensor on a friction cam for a motorized surgical handle assembly configured in accordance with a number of embodiments of the present disclosure.

FIG. 23 shows an example of a sensor on a circuit board for a motorized surgical handle assembly configured in accordance with a number of embodiments of the present disclosure.

FIG. 24 is a flowchart illustrating a method for operating a motorized surgical handle assembly apparatus in accordance with a number of embodiments of the present disclosure.

### DETAILED DESCRIPTION

The present disclosure includes apparatuses for motorized or power surgical handle assemblies and surgical handles. An example apparatus includes a reloadable cartridge assembly and a motorized surgical handle assembly. The surgical handle includes a movable handle that allows for the clamping (e.g., grasping) and unclamping (e.g., ungrasping) of a reloadable cartridge assembly prior to delivering staples.

In a number of embodiments, the reloadable cartridge assembly can include a first elongated member and a second elongated member. The first elongated member and the second elongated member can sometimes be referred to as jaws. The jaws can be used to clamp and/or grasp tissue. One of the elongated members can house one or more staple cartridges. The other elongated member can have an anvil that can be used to form a staple when driven from the staple cartridge. In a number of embodiments, the motorized surgical handle assembly has a pawl that interfaces with a gear rack to move the gear rack proximally and distally to clamp (e.g., close) and/or unclamp (e.g., open) the jaws.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the art to practice the embodiments of this disclosure, and it is to be understood that other embodiments may be utilized and that process, electrical, and structural changes may be made without departing from the scope of the present disclosure.

It is to be understood that the terminology used herein is for the purpose of describing particular embodiments only and is not intended to be limiting. As used herein, the singular forms “a”, “an”, and “the” can include both singular and plural referents, unless the context clearly dictates otherwise. In addition, “a number of”, “at least one”, and “one or more” (e.g., a number of bosses) can refer to one or more bosses, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to”. The terms “coupled” and “coupling” mean to be directly or indirectly connected physically or for access to and movement of the movable handle member, as appropriate to the context.

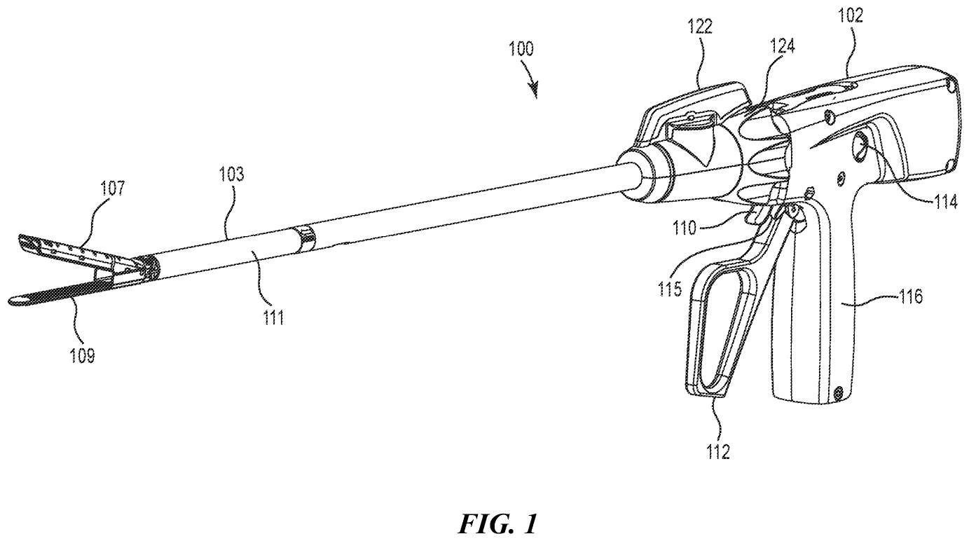

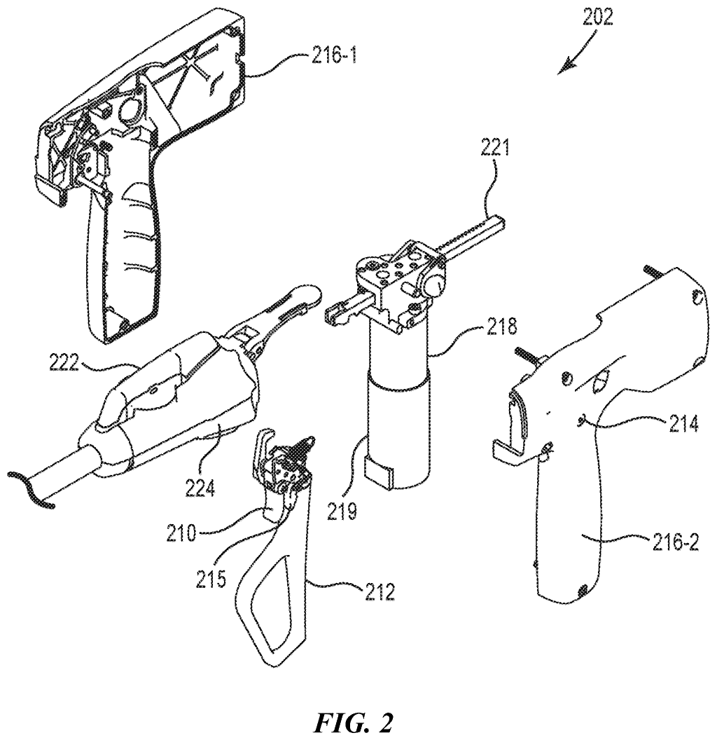

The figures herein follow a numbering convention in which the first digit or digits correspond to the figure number and the remaining digits identify an element or component in the figure. Similar elements or components between different figures may be identified by the use of similar digits. For example, 102 may reference element “2” in FIG. 1, and a similar element may be referenced as 202 in FIG. 2. As will be appreciated, elements shown in the various embodiments herein can be added, exchanged, and/or eliminated so as to provide a number of additional embodiments of the present disclosure. In addition, the proportion and/or the relative scale of the elements provided in the figures are intended to illustrate certain embodiments of the present disclosure and should not be taken in a limiting sense.

FIG. 1 is a schematic diagram of an apparatus 100 including a motorized surgical handle assembly 102 and a reloadable cartridge assembly 103 in accordance with a number of embodiments of the present disclosure. In the example, the apparatus 100 can be a surgical stapler, for example.

As shown in the example of FIG. 1, the reloadable cartridge assembly 103, e.g., a disposable loading unit, can be releasably secured to a distal end of an elongated body of the motorized surgical handle assembly 102. In this example, the reloadable cartridge assembly 103 can include a cartridge shaft 111, a first elongated member 107 and a second elongated member 109 that can be used to clamp tissue. One of the elongated members can house one or more staple cartridges. The other elongated member can have an anvil that can be used to form a staple when driven from the staple cartridge. Apparatus 100 can receive reloadable cartridge assemblies having rows of staples. In a number of embodiments, third party reloadable cartridge and/or reloadable cartridge assemblies may be used with the motorized surgical handle assembly 102 and embodiments of motorized surgical handle assembly 102 may be configured to receive the same.

The motorized surgical handle assembly 102 can include a radial positioner 124, an articulation assembly activated by articulation knob 122, and non-movable handle (e.g., stationary handle) 116. The reloadable cartridge assembly 103 can be actuated using articulation knob 122 and/or radial positioner 124 to reach a stapling site. Radial positioner 124 rotates the reloadable cartridge assembly. Articulation knob 122 positions the elongated members 107 and 109 at a particular angle for stapling. The articulation knob 122 can be configured to actuate rotationally and the reloadable cartridge assembly 103 can rotate about an axis of a particular plane in response to the articulation knob 122 being actuated rotationally by a user. Movable handle 112 can be used to clamp and unclamp elongated members 107 and 109.

Power trigger 110 can be used to activate the electric motor to move the gear rack distally. Safety switch 115 may be used to allow power to flow to the electric motor and/or block the power trigger 110 from being activated. Reverse (e.g., retract) button 114 can be used to activate the electric motor to move the gear rack proximally.

FIG. 2 is a schematic diagram of a motorized surgical handle assembly 202 in accordance with a number of embodiments of the present disclosure. A distal portion of the motorized surgical handle assembly 202 can include the articulation knob 222 and radial positioner 224. Not shown is a driveshaft that cooperates with gear rack 221 to effect movement for a reloadable cartridge assembly (e.g., reloadable cartridge assembly 103 in FIG. 1). FIG. 2 illustrates a first handle half 216-1 and a second handle half 216-2 which provide a non-movable handle for the user of the device and houses a drivetrain 218. The first handle half 216-1 and/or the second handle half 216-2 may also include components that are used in the clamping and/or unclamping of the jaws. In some embodiments, drivetrain 218 can include a number of gears, not shown, and/or a power source such as a battery and/or an electric motor 219. In other embodiments, drivetrain 218 can include an electric motor and a number of gears and a battery can be located elsewhere such as between the first handle half 216-1 and the second handle half 216-2. In some embodiments the handle can include a power cord such that the electric motor can be powered by AC or DC current supplied from outside the device. The gear rack 221 can interact with the drivetrain 218 and a drive shaft, not shown, can be coupled to a distal end of the gear rack 221. Power trigger 210 and/or reverse button 214 can be used to activate the drivetrain 218, movable handle 212 can be used to clamp and unclamp the elongated members, and safety switch 215 can allow power to flow to the electric motor 219 and/or block the power trigger 210 from being activated.

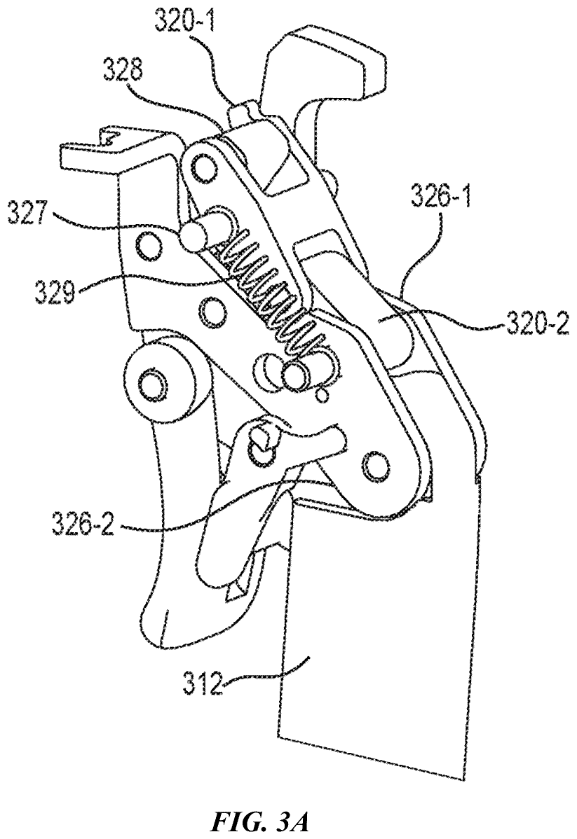

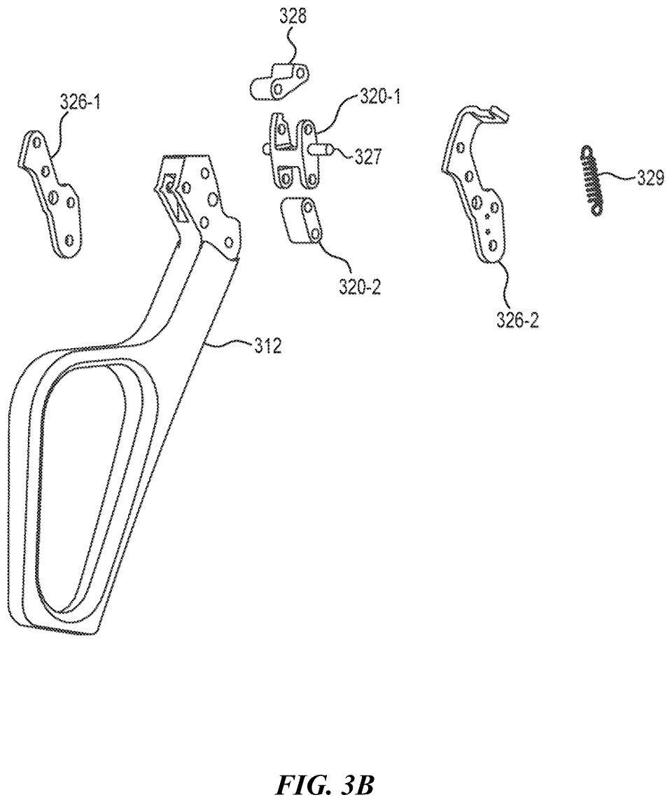

FIG. 3A is a schematic diagram of an embodiment of a movable handle 312 in accordance with a number of embodiments of the present disclosure. The movable handle 312 can include a first side plate 326-1, a second side plate 326-2, a first linkage 320-1, a second linkage 320-2, a pawl 328, a disengagement pin 327, and a linkage spring 329.

FIG. 3B is a schematic diagram of an exploded view of a movable handle 312 in accordance with a number of embodiments of the present disclosure. The movable handle 312 can include a first side plate 326-1, a second side plate 326-2, a first linkage 320-1, a second linkage 320-2, a pawl 328, a disengagement pin 327, and a linkage spring 329.

FIG. 4 is a schematic diagram of a first handle half 416-1 in accordance with a number of embodiments of the present disclosure. The first handle half 416-1 can include and/or be coupled to a movable handle lock spring 430, a clamp lock 432, a first disengagement insert 434-1, and a reset spring 436.

FIG. 5 is a schematic diagram of a second handle half 516-2 in accordance with a number of embodiments of the present disclosure. The second handle half 516-2 can include a second disengagement insert 534-2.

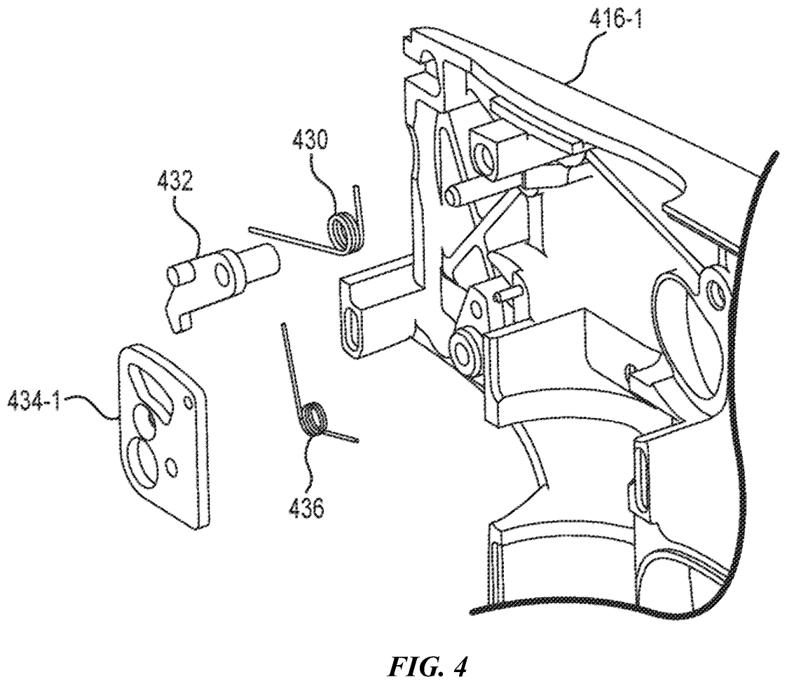

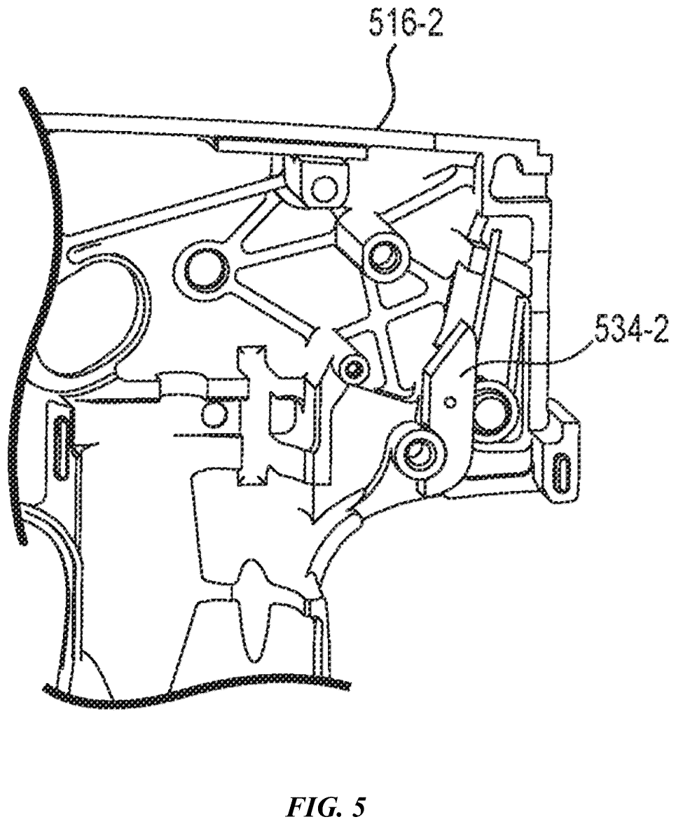

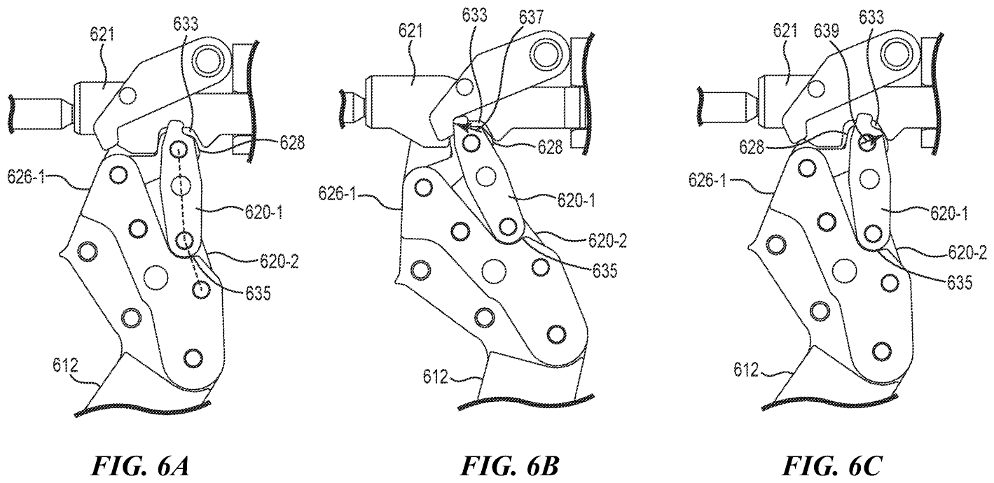

FIGS. 6A, 6B, and 6C are schematic diagrams of a mechanism for grasping and ungrasping a reloadable cartridge assembly (e.g., reloadable cartridge assembly 103 in FIG. 1) in accordance with a number of embodiments of the present disclosure. FIG. 6A illustrates an example of the movable handle 612 in a clamping state. In this view, pawl 628 is in gear rack notch 633 of gear rack 621. A first linkage 620-1 and a second linkage 620-2 can be relatively aligned and positioned adjacent to side plate 626-1. While the description uses the phrase ‘relatively aligned,’ the alignment is not critical. In the state shown in FIGS. 6A, 6B, and 6C, as movable handle 612 is moved proximally, pressure is exerted through the second linkage 620-2 and through the first linkage 620-1 to keep pawl 628 in gear rack notch 633. Also as shown, a pivot point junction 635 between the second linkage 620-2 and the first linkage 620-1 is pressing against movable handle 612. As the first linkage 620-1 and the second linkage 620-2 cannot move, the force is transmitted to the pawl 628. Also, as shown in FIG. 3A, linkage spring 329 is attached at one end to a lower attachment point of the second linkage 320-2 and at the other end to disengagement pin 327. The force of linkage spring 329 will attempt to move the first linkage 320-1 and the second linkage 320-2 out of alignment. Thus, when the first linkage 620-1 and the second linkage 620-2 are as shown in FIG. 6A, the first linkage 620-1 and/or the second linkage 620-2 can stay in a slightly over-center position as they try to overcome the force of the linkage spring (e.g., linkage spring 326 in FIG. 3A) to go over-center in the other direction. At many places in this disclosure, components are shown as being separate. The inventions disclosed here also cover embodiments where multiple components are integrated together into a single component.

In FIG. 6B, as a user actuates movable handle 612 proximally, pawl 628 pushes on the distal end of gear rack notch 633, as shown by arrow 637, moving gear rack 621 in a distal direction. This distal movement moves a drive assembly including the gear rack 621, a drive shaft, not shown, of a motorized surgical handle assembly (e.g., motorized surgical handle assembly 102 and 202 in FIG. 1 and FIG. 2, respectively), and a blade shaft, not shown, of a reloadable cartridge assembly (e.g., reloadable cartridge assembly 103 in FIG. 1) distally. In some embodiments, the distal end of the drive assembly comprises an I-beam. As the I-beam moves distally, the top and bottom sections of the I-beam will force elongated members (e.g., elongated members 107 and 109 in FIG. 1) to move toward each other in a clamping motion.

In FIG. 6C, as a user actuates movable handle 612 distally, pawl 628 pushes on the proximal end of gear rack notch 633, as shown by arrow 639, moving gear rack 621 in a proximal direction. This proximal movement moves the entire drive assembly including a drive shaft, not shown, of a surgical handle assembly (e.g., motorized surgical handle assembly 102 and 202 in FIG. 1 and FIG. 2, respectively), a blade shaft, not shown, of a reloadable cartridge assembly (e.g., reloadable cartridge assembly 103 in FIG. 1), and an I-beam, not shown, proximally. In some embodiments, as the I-beam moves proximally, the top and bottom sections of the I-beam will move away from interacting with elongated members (e.g., elongated members 107 and 109 in FIG. 1) which will allow the elongated members to move away from each other in an opening or unclamping motion.

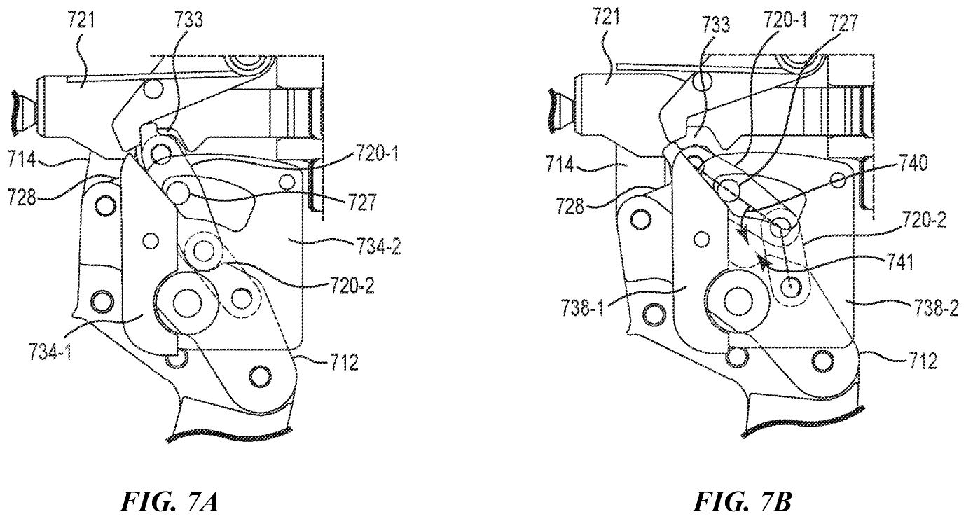

FIGS. 7A and 7B are schematic diagrams of a disengagement mechanism in accordance with a number of embodiments of the present disclosure. When the user has satisfactorily clamped the elongated members (e.g., elongated members 107 and 109 in FIG. 1) and they are placed in the desired position, further proximal movement of movable handle 712 will rotate pawl 728 out of gear rack notch 733. As seen in FIG. 7A, further proximal movement of movable handle 712 will cause disengagement pin 727 to contact a first disengagement insert 734-1 and a second disengagement insert 734-2. This contact will force the bottom of a first linkage 720-1 and a top of second linkage 720-2 to move away from movable handle 712. Arrows 740 and 741, as illustrated in FIG. 7B, show the middle of the first linkage 720-1 and the middle of the second linkage 720-2 moving toward each other. This movement pulls pawl 728 away from and out of gear rack notch 733 in gear rack 721. When movable handle 712 is in the locked position, the elongated members will stay in the clamped position. In some embodiments, the surgical handle assembly will not include a first insert 734-1 and the second insert 734-2 can cause the movement discussed earlier in this paragraph.

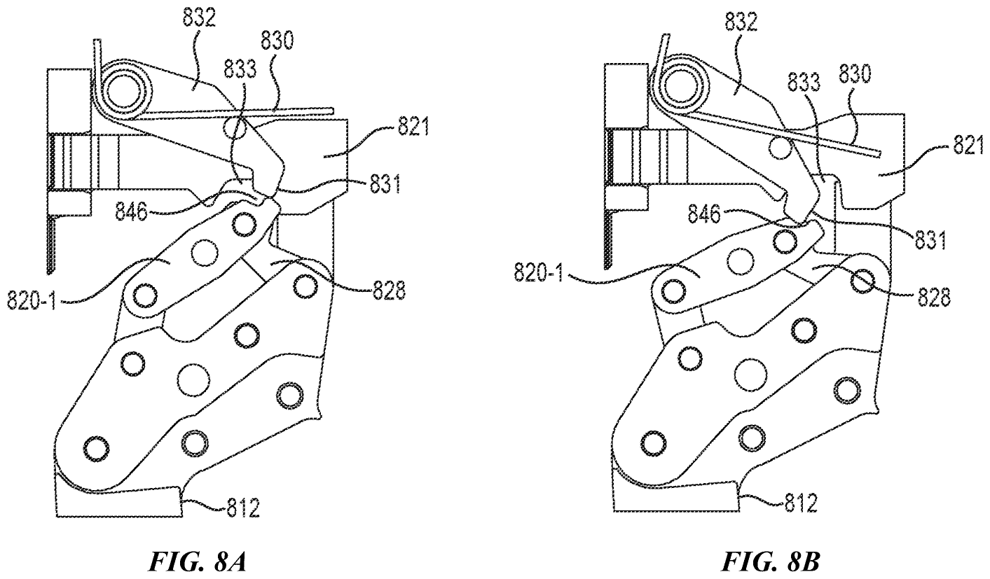

FIGS. 8A and 8B are schematic diagrams of a locking mechanism for a movable handle 812 in accordance with a number of embodiments of the present disclosure. As shown in FIG. 8A, movable handle lock spring 830 tensions clamp lock 832 against a first linkage 820-1. As the first linkage 820-1 is rotated, pawl 828 moves out of gear rack notch 833 and clamp lock 832 is rotated toward the first linkage 820-1 until a part of clamp lock 832, protrusion 831, is positioned in locking notch 846 of the first linkage 820-1. This is shown in FIG. 8B. The rotation of the pawl 828 and first linkage 820-1 is stopped by contact between the pawl 828 and the movable handle 812. The movable handle 812 is now locked in the proximal position. In this position, none of the components of the clamping/unclamping mechanism will interfere with the movement of gear rack 821 and the elongated members (e.g., elongated members 107 and 109 in FIG. 1) will stay in the clamped position.

Once the movable handle 812 is locked in the clamped position, the user may deliver some or all of the staples in a staple cartridge. This is accomplished by first activating a safety switch (e.g., safety switch 115 in FIG. 1) and then activating a power trigger (e.g., power trigger 210 in FIG. 2) which activates the drivetrain (e.g., drivetrain 218 in FIG. 2) to effect distal movement of the gear rack 821. Distal movement of a drive assembly causes an I-beam to advance through the elongated members and to deliver staples. The I-beam also comprises a cutting edge on the distal end so that stapled tissue is cut. At any time during a procedure, including immediately after locking the movable handle 812, after delivering some staples, or after delivering all the staples, a user may desire to unclamp the elongated members and/or retract the I-beam. To do this, the user activates reverse or retract button (e.g., retract button 114 in FIG. 1). The activation of the retract button causes a drivetrain (e.g., drivetrain 218 in FIG. 2) to drive gear rack 821 in a proximal direction moving the I-beam away from the elongated members, allowing the elongated members to open or unclamp. At this stage the movable handle 812 is allowed to spring to its distal position. Now, the user can either clamp the elongated members or replace the reloadable cartridge.

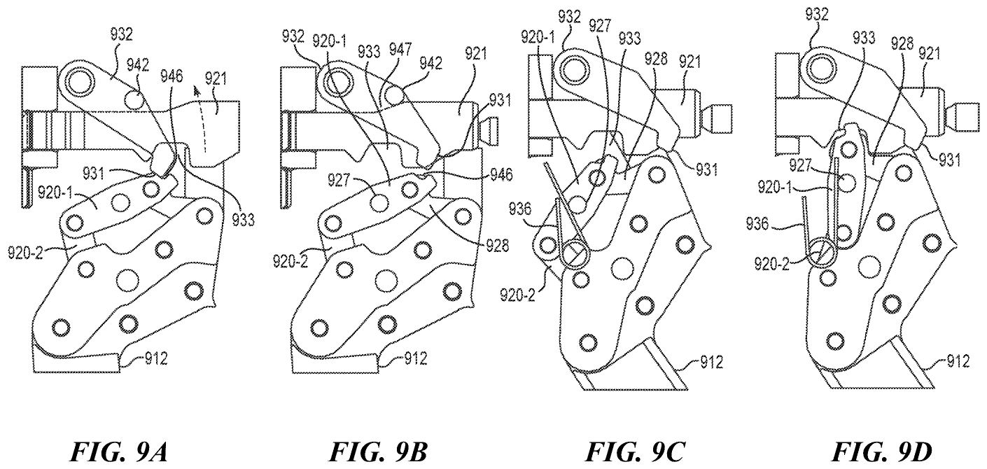

FIGS. 9A, 9B, 9C, and 9D are schematic diagrams of a reset mechanism for a movable handle 912 in accordance with a number of embodiments of the present disclosure with 9A and 9B showing the unlocking of the movable handle 912 and 9C and 9D showing the resetting of pawl 928 back into gear rack notch 933 as the handle opens. FIG. 9A shows cam lock 932 positioned within locking notch 946 of first linkage 920-1.

As shown in FIG. 9B, when a user activates retract button (e.g., retract button 114 in FIG. 1) causing a drivetrain to move a gear rack 921 in a proximal direction, lock cam 942, positioned on clamp lock 932, will interact with cam surface 947 of gear rack 921 when the gear rack 921 is almost fully retracted. This interaction will cause clamp lock 932 and protrusion 931 to rotate out of locking notch 946 of a first linkage 920-1 allowing the movable handle 912 to move distally. Although not illustrated, in a number of embodiments, the lock cam 942 can be positioned on gear rack 921 and can interact with a cam surface of clamp lock 932 to cause clamp lock 932 and protrusion 931 to rotate out of locking notch 946 allowing the movable handle 912 to move distally.

As shown in FIG. 9C, after movable handle 912 has moved in the distal direction, reset spring 936 exerts force on disengagement pin 927 which causes disengagement pin 927 to move toward movable handle 912 and pawl 928 into gear rack notch 933. In some examples, a cam surface on one or both of the handle halves (e.g., handle halves 216-1 and 216-2 in FIG. 2) can be used instead of or in tandem with the reset spring 936. For example, the disengagement pin can contact the cam surface and the cam surface can push the disengagement pin up as the movable handle 912 continues to open.

FIG. 9D shows the assembly after being reset. Now, pawl 928 is positioned in gear rack notch 933 of gear rack 921 and a first linkage 920-1 and a second linkage 920-2 are relatively aligned and slightly over-center. At this position, a user can either clamp elongated members (e.g., elongated members 107 and 109 in FIG. 1) or can change a reloadable cartridge.

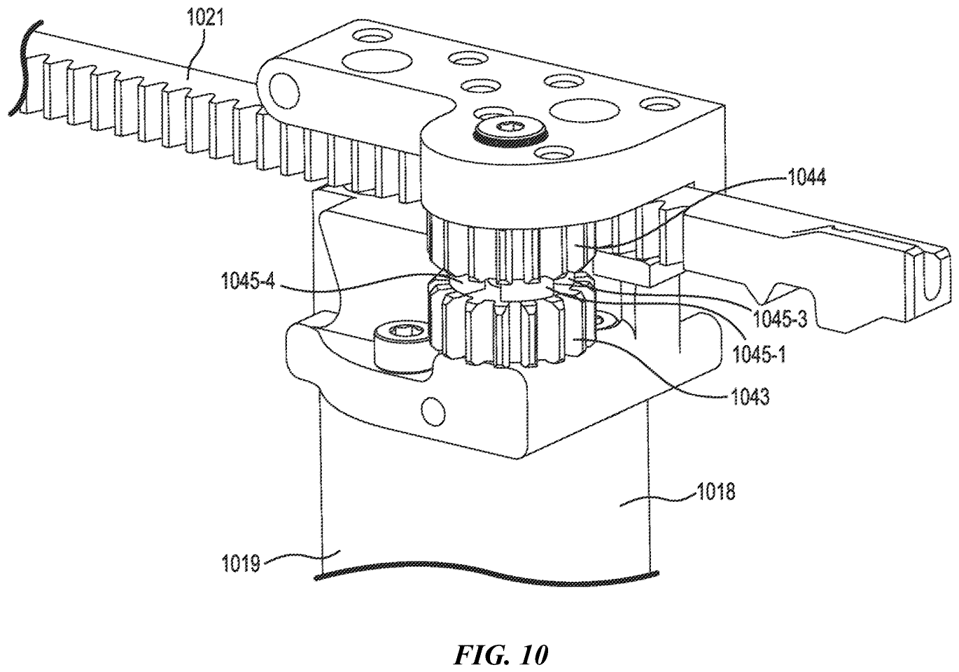

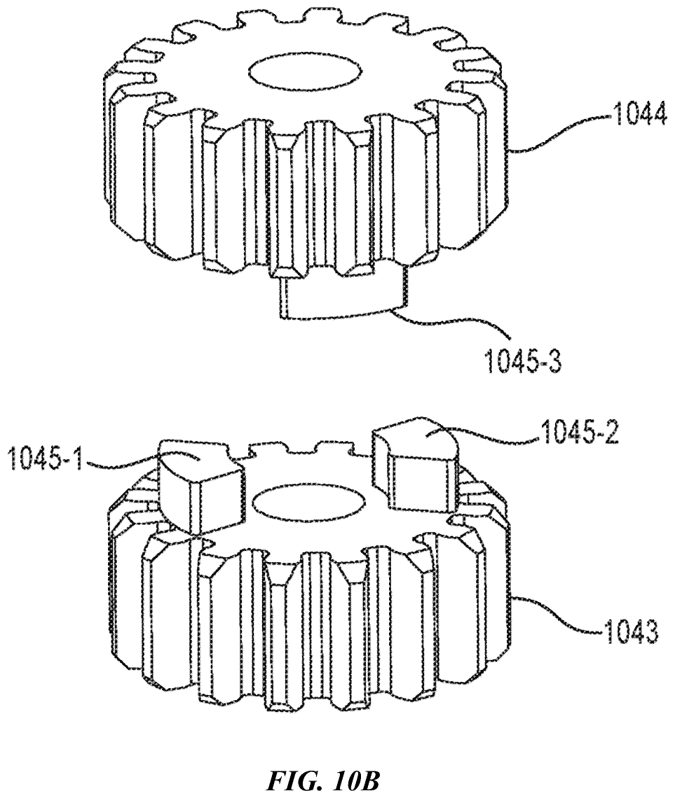

FIGS. 10A and 10B are schematic diagrams of a drivetrain 1018 in accordance with a number of embodiments of the present invention. Near the top of drivetrain 1018 is a gear system that includes driving gear 1043 and driven gear 1044. The electric motor 1019 rotates driving gear 1043 (cither directly or through one or more other gears (not shown)) through the teeth on its circumferential surface and, through the interaction of bosses 1045-1 and/or 1045-2 with bosses 1045-3 and/or 1045-4, driving gear 1043 rotates driven gear 1044. Driven gear 1044 drives gear rack 1021 through its teeth on its circumferential surface. In some embodiments this will be referred to as a rack and pinion system. The rack, or gear rack 1021, moves in response to pinion or driven gear 1044 rotation as the cogs or teeth of driven gear 1044 interact with the cogs or teeth of gear rack 1021. In some embodiments, driven gear 1044 is associated with gear rack 1021 such that rotation of driven gear 1044 causes linear movement of gear rack 1021.

Shown in FIG. 10B are two bosses 1045-1 and 1045-2 positioned on top of driving gear 1043. Driven gear 1044 also has two bosses with boss 1045-3 and 1045-4 shown in the FIG. 10A. As electric motor 1019 rotates driving gear 1043, it will rotate until bosses 1045-1 and/or 1045-2 touch bosses 1045-3 and/or 1045-4. Further rotation of driving gear 1043 will cause rotation of driven gear 1044 and movement of gear rack 1021. The spacing between each of the bosses 1045-1, 1045-2, 1045-3, and 1045-4 allow for an amount of ‘play’ in the drivetrain system. Either driving gear 1043 or driven gear 1044 can rotate an amount without causing rotation of the other gear. The amount of play can be controlled by changing the width in the circumferential direction of the gears 1043 and 1044 of the bosses 1045-1, 1045-2, 1045-3, and 1045-4. Narrower bosses 1045-1, 1045-2, 1045-3, and 1045-4 will allow for more movement or play than wider bosses 1045-1, 1045-2, 1045-3, and 1045-4. While two bosses are shown on each gear, one or more bosses 1045-1, 1045-2, 1045-3, and 1045-4 could be used on one or both gears 1043 and 1044. As discussed herein, when movable handle (e.g., movable handle 112 in FIG. 1) is moved proximally and distally to clamp and unclamp elongated members (e.g., elongated members 107 and 109 in FIG. 1), this play in the system allows the gear rack 1021 and driven gear 1044 to move an amount sufficient to clamp or unclamp the elongated members without causing rotation of driving gear 1043. With some systems, rotation of driving gear 1043 will be very difficult due to the high gear reduction in electric motor 1019. In some embodiments, electric motor 1019 is described as being connected to driving gear 1043. In some embodiments, there is an additional gear system, not shown, in between the output of the electric motor 1019, generally a rotating shaft, and driving gear 1043. In some embodiments, gears 1043 and 1044 are identical, they are just mounted in a mirror image fashion. While gears 1043 and 1044 are shown with bosses 1045-1, 1045-2, 1045-3, and 1045-4 that extend away from a surface of the gears 1043 and 1044, in some embodiments a first gear of the gears 1043 or 1044 can have at least one boss of one or more bosses 1045-1, 1045-2, 1045-3, and 1045-4 that extends away from the surface while a second gear of the gears 1044 or 1043 can have a depression in the surface that will interact with the at least one raised boss of the first gear. In some embodiments, the width of the bosses can be balanced as thinner cogs enable larger potential movement of one gear relative to the other gear without needing to overcome the force of drivetrain 1018 but a boss that is too thin could be sheared off In some embodiments, the number of and thickness of bosses (or depression(s)) will dictate the relative movement of a first gear relative to a second gear before the second gear will begin to move, but in all embodiments this distance, or amount of ‘play,’ will be less than 360°. In embodiments where each gear has only one boss, the amount of play may be between 250° to 290°. In embodiments where each gear has two bosses, the play may be in the range of 60° to 100° or, in some embodiments, about 70°.

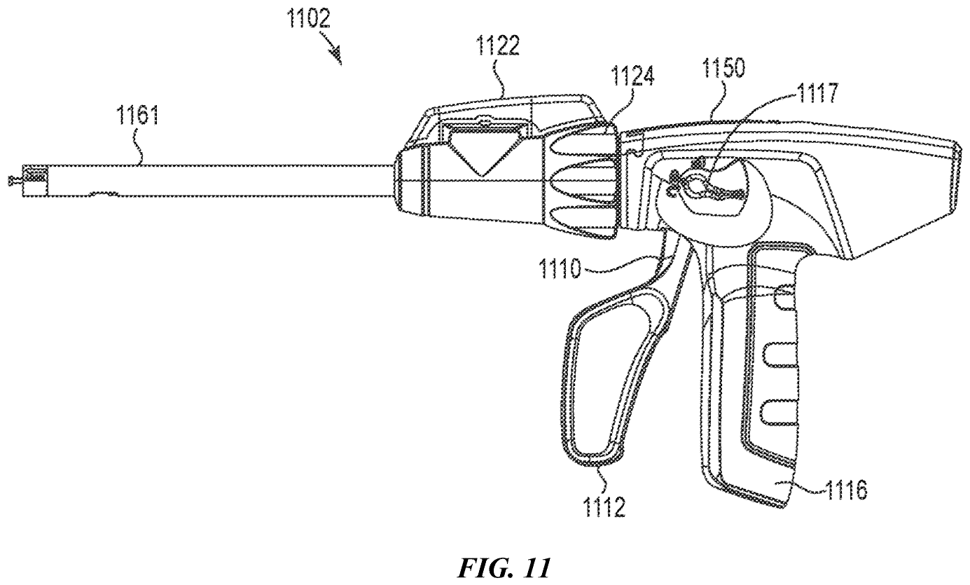

FIG. 11 is a schematic diagram of a motorized surgical handle assembly 1102 in accordance with a number of embodiments of the present disclosure. In use, motorized surgical handle assembly 1102 can be attached to reloadable cartridge assembly (e.g., reloadable cartridge assembly 103 in FIG. 1). As shown in the example of FIG. 11, the motorized surgical handle assembly 1102 can include a radial positioner 1124, an articulation assembly activated by articulation knob 1122, non-movable handle 1116, outer shaft 1161, and manual retraction or bailout handle 1150. Movable handle 1112 can be used to clamp and unclamp elongated members (e.g., elongated members 107 and 109 in FIG. 1). Power trigger 1110 can be used to activate the electric motor to move the gear rack (e.g., gear rack 1021 in FIG. 10A) distally.

Selector lever 1117 can include a number of settings, which can include a locked position (e.g., safe), an unlocked position (e.g., fire), and a reverse position. While the selector lever 1117 is in the locked position, movable handle 1112 may be used to clamp and unclamp the jaws but power trigger 1110 is electrically deactivated. When the selector lever 1117 is in the unlocked position, power trigger 1110 may be used to allow power to flow to an electric motor. As will be discussed later, movable handle 1112 needs to be in a proximal position before the selector lever 1117 can be moved from the locked position to the unlocked position. When set to the reverse (e.g., retract) position which, in some embodiments, is a momentary position, selector lever 1117 will cause the electric motor to be activated to move the gear rack proximally.

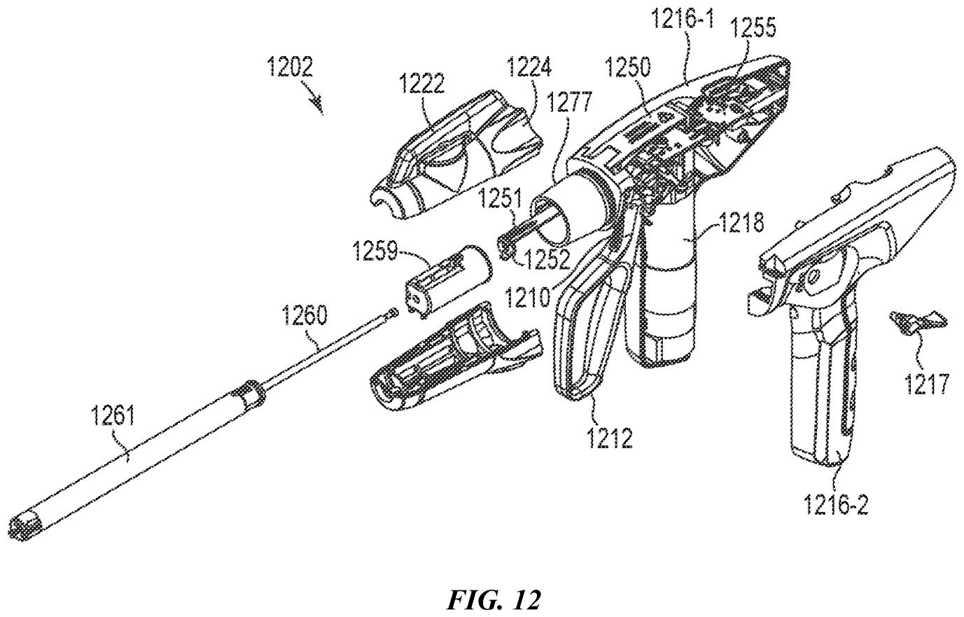

FIG. 12 is a schematic diagram of an exploded view of a motorized surgical handle assembly 1202 in accordance with a number of embodiments of the present disclosure. A distal portion of the motorized surgical handle assembly 1202 can include the articulation knob 1222 and radial positioner 1224. Drive shaft (e.g., drive rod) 1260 cooperates with the gear rack (e.g., gear rack 1021 in FIG. 10A) to effect movement for a reloadable cartridge assembly (e.g., reloadable cartridge assembly 103 in FIG. 1).

FIG. 12 illustrates a second handle half 1216-2 which, along with first handle half 1216-1, provides a non-movable handle (e.g., non-movable handle 1116 in FIG. 11) for the user of the motorized surgical handle assembly 1201, houses a drivetrain 1218, and encases the electrical and mechanical mechanisms. The first handle half 1216-1 and/or the second handle half 1216-2 may also include components that are used in the clamping and/or unclamping of the jaws.

In some embodiments, drivetrain 1218 can include a number of gears, not shown, and/or a power source such as a battery and/or an electric motor that can be battery powered or connected to an external power source, such as, an alternating current (AC) source. In other embodiments, drivetrain 1218 can include an electric motor, a number of gears, and/or a battery that can be located elsewhere, such as, between the first handle half 1216-1 and the second handle half 1216-2. A gear rack (e.g., gear rack 1021 in FIG. 10A) can interact with the drivetrain 1218 and a drive shaft 1260 can be coupled to a distal end of the gear rack.

Power trigger 1210 and selector lever 1217 can be used to activate the drivetrain 1218. Movable handle 1212 can be used to clamp and unclamp the elongated members, and selector lever 1217 can allow power to flow to the electric motor and/or block the power trigger 1210 from being activated. Also shown in FIG. 12 is outer shaft 1261, nose cone 1277, bailout handle 1250, circuit board 1255, articulation lock 1259, toggle plates 1252, and bailout bar 1251.

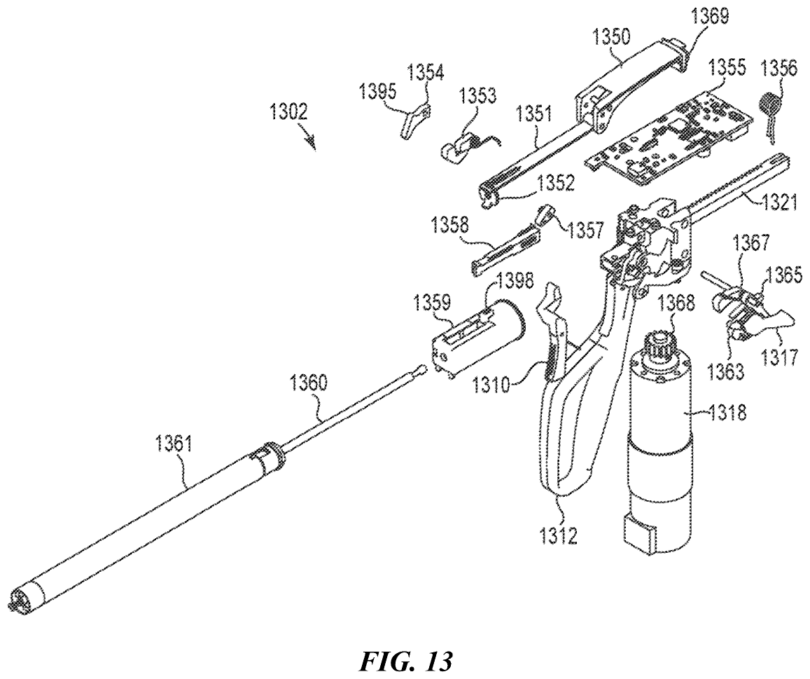

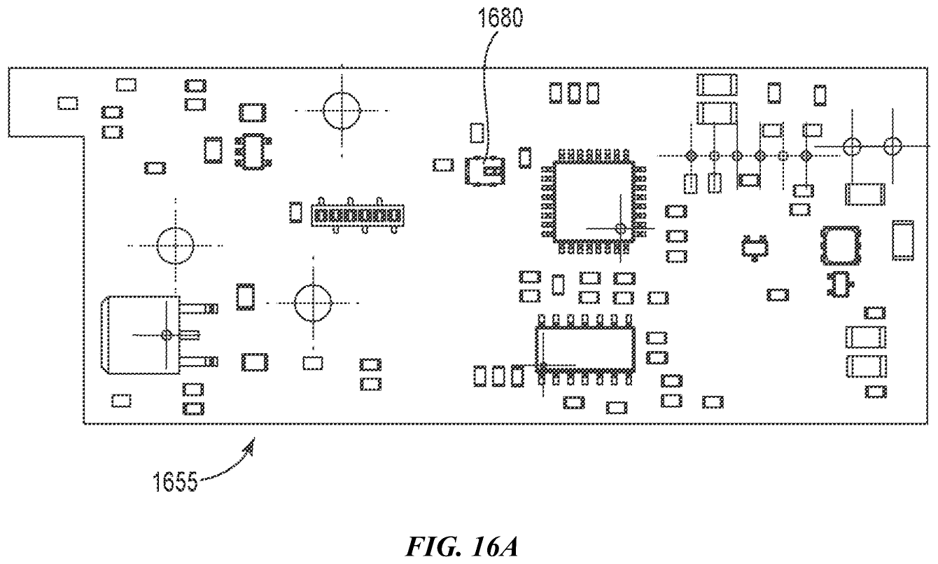

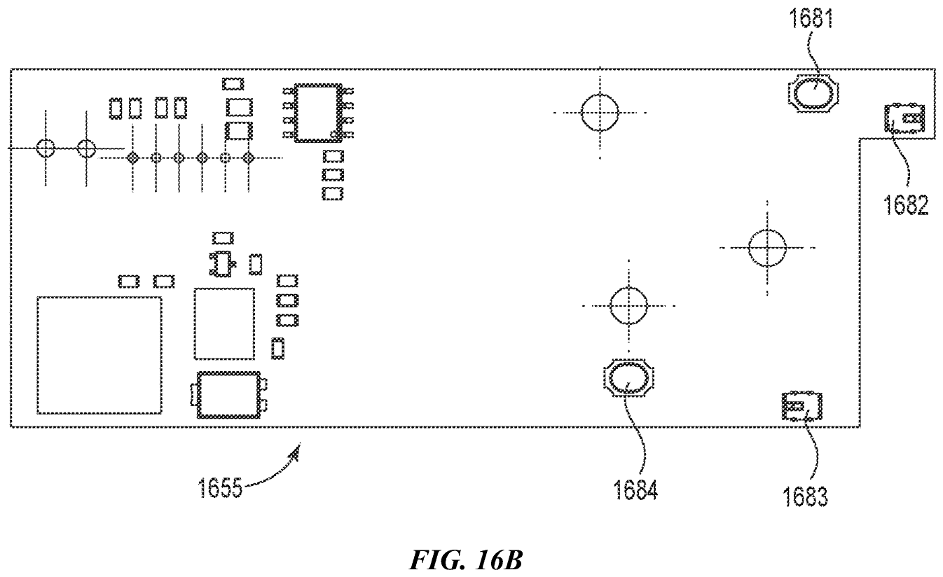

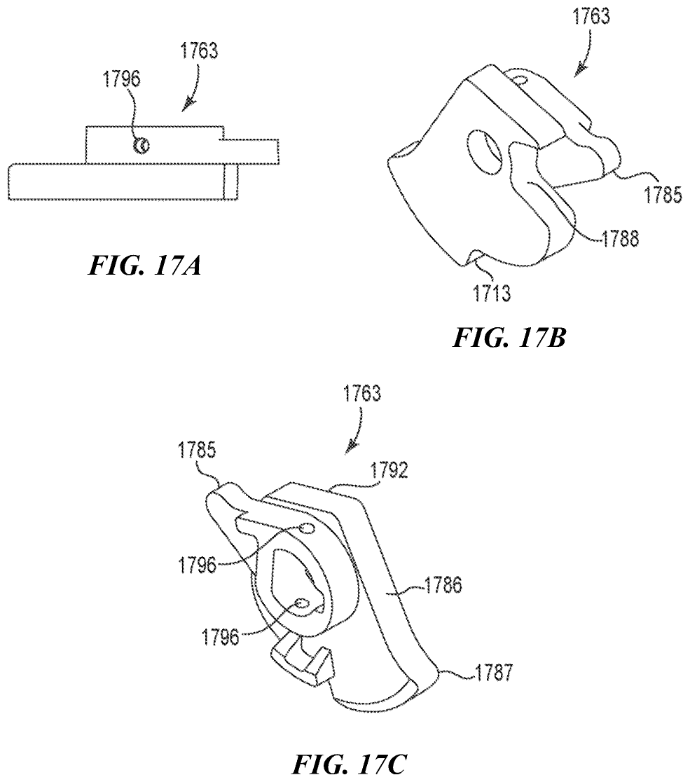

FIG. 13 is a schematic diagram of an exploded view of a motorized surgical handle assembly 1302 in accordance with a number of embodiments of the present disclosure. Selector lever 1317 is non-rotationally fixed to selector cam 1363. When selector lever 1317 is rotated to the unlocked position, which can only happen when movable handle 1312 is moved to its most proximal position and the jaw assembly is closed, selector cam 1363 rotates and protrusion (e.g., protrusion 1785 in FIGS. 17B and 17C) activates a switch (e.g., switch 1683 in FIG. 16B) on circuit board 1355. When the switch is activated, energy is allowed to flow to drivetrain 1318.

When selector lever 1317 is rotated to the reverse position, selector cam 1363 rotates reverse cam 1365 and causes protrusion 1497 on reverse cam 1365 to activate reverse switch 1684 on circuit board 1355 and causes drivetrain 1318 to move in reverse to retract gear rack 1321 and drive shaft 1360. When activated, drivetrain 1318 causes gear 1368 to rotate. Gear 1368 is configured to cause driving gear (e.g., gear 1043 in FIGS. 10A and 10B) to rotate and move gear rack 1321. The proximal end of drive shaft 1360 extends through articulation lock 1359 and is connected to the distal end of gear rack 1321. A portion of drive shaft 1360 is housed within outer shaft 1361.

When gear rack 1321 is fully reversed, retraction spring 1356 will be compressed and a control system will experience a spike in current as the gear rack 1321 is positioned against retraction spring 1356 and other components of the motorized surgical handle assembly 1302. At this point drivetrain 1318 will change direction and rotate the driving gear to unload retraction spring 1356, which causes the gear rack 1321 to move in the distal direction a short distance, for example 0.5, 1, 1.2, or 2 mm. This forward movement unloads retraction spring 1356 and will position bosses (e.g., bosses 1045-1 and 1045-2 in FIG. 10B) of driving gear (e.g., driving gear 1043 in FIGS. 10A and 10B) between bosses (e.g., bosses 1045-3 and 1045-4 in FIG. 10A) of driven gear (e.g., driven gear 1044 in FIGS. 10A and 10B) so that the clamp and unclamp mechanism will work.

When movable handle 1312 is moved to the proximal most position the frictional force of friction cam 1353 and its associated spring on movable handle 1312 will hold movable handle 1312 in the proximal most position. A user/physician can open the jaws by moving the movable handle 1312 distally manually, by forcing the movable handle 1312 distally, or by using the reverse function of the selector lever 1317.

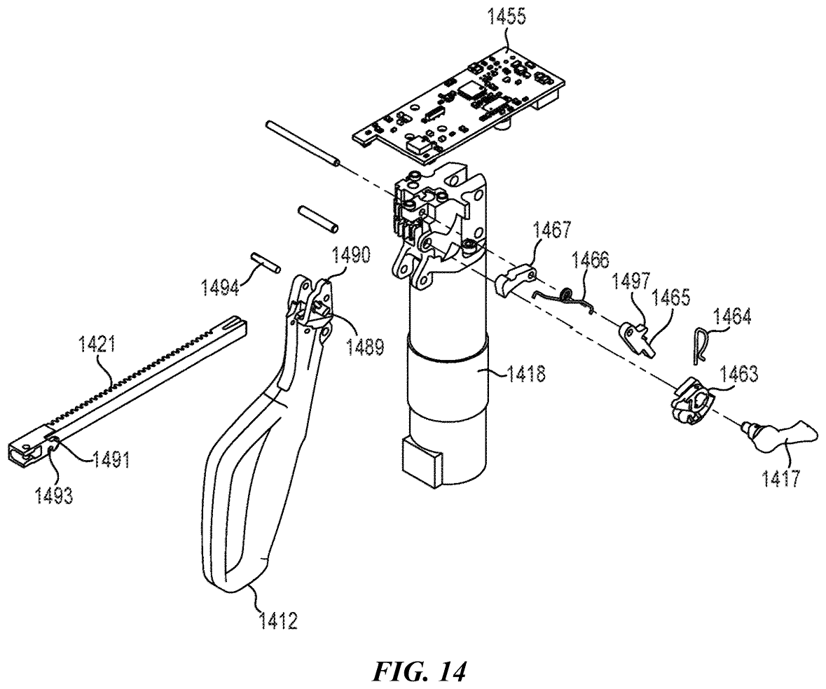

When the selector lever 1317 is rotated to the unlocked position, surface (e.g., surface 1788 in FIG. 17B) of selector cam 1363 will rotate against a locking protrusion (e.g., locking protrusion 1489 in FIG. 14) of movable handle 1312 and lock movable handle 1312 in the proximal position. After the gear rack 1321 begins to travel distally, locking cam 1367 will interact with a notch (e.g., notch 1490 in FIG. 14) of movable handle 1312 and lock movable handle 1312 in the proximal position.

When a reload unit, such as reloadable cartridge assembly 103 in FIG. 1, is attached to the distal end of the motorized surgical handle assembly 1302, a bar, not shown, connected to the distal end of articulation lock 1359 will move proximally and cause articulation lock 1359 to move proximally. Proximal movement of articulation lock 1359 moves bar 1358 which moves cam 1357 into contact with (e.g., closes) reload switch (e.g., reload switch 1682 in FIG. 16B) on circuit board 1355 which allows drivetrain 1318 to be activated. If there is no reloadable cartridge assembly attached to the motorized surgical handle assembly 1302, drivetrain 1318 cannot be activated.

Protrusion 1398 on articulation lock 1359 is configured to interact with an articulation mechanism of motorized surgical handle assembly 1302 such that articulation knob (e.g., articulation knob 1222 in FIG. 12) cannot be rotated if a reloadable cartridge assembly is not attached to the motorized surgical handle assembly 1302. When a reloadable cartridge assembly is attached, the movable handle 1312 is in the proximal most position, selector lever 1317 is switched to an unlocked position, and a user moves power trigger 1310 proximally, trigger 1310 pushes intermediate lever 1354, specifically protrusion 1395, into contact with a power switch (e.g., power switch 1681 in FIG. 16B) on circuit board 1355 causing the drivetrain 1318 to move gear rack 1321 in a distal direction.

Friction cam 1353 is configured to provide friction to movable handle 1312 so that the handle is less likely to move without a user moving it in a proximal or distal direction. Bailout handle 1350 includes u-shaped latch 1369. U-shaped latch 1369 is configured to interact with the first handle half (e.g., first handle half 1216-1 in FIG. 12) and the second handle half (e.g., second handle half 1216-2 in FIG. 12) to keep bailout handle 1350 secured in the down position. The bottom of the u-shaped latch 1369 interacts with switch (e.g., switch 1680 in FIG. 16A) on circuit board 1355. When the switch is closed (e.g., pressed), power is supplied to the circuit board 1355. When bailout handle 1350 is lifted and u-shaped latch 1369 is not in contact with switch 1680 (e.g., the switch is open), no power will flow to the circuit board 1355. Additional functions and uses of bailout handle 1350, bailout bar 1351, and toggle plates 1352 will be explained elsewhere.

FIG. 14 is a schematic diagram of aspects of a selector lever 1417 of a motorized surgical handle assembly (e.g., motorized surgical handle assembly 1302 in FIG. 13) in accordance with a number of embodiments of the present disclosure. Selector lever 1417 is non-rotationally attached to selector cam 1463 by pin 1464. In some embodiments, selector lever 1417 and selector cam 1463 can be a single piece and pin 1464 will not be needed. When assembled, selector cam 1463 rotates with selector lever 1417. When selector lever 1417 is rotated to the unlocked position, protrusion (e.g., protrusion 1785 in FIGS. 17B and 17C on selector cam 1463 rotates into contact with (e.g., closes) switch (e.g., switch 1683 in FIG. 16B) on circuit board 1455 putting a movable handle 1412 into the ready to fire position.

When selector lever 1417 is rotated to a reverse position, reverse cam 1465 is rotated by selector cam 1463 so that protrusion 1497 moves into contact with (e.g., closes) reverse switch (e.g., reverse switch 1684 in FIG. 16B) on circuit board 1455 and causes drivetrain 1418 to move gear rack 1421 in a proximal direction. Spring 1466 is configured so that reverse cam 1465, selector cam 1463, and selector lever 1417 immediately move back to the locked position after the physician/user stops exerting force on selector lever 1417 after having turned selector lever 1417 to the reverse position.

Locking cam 1467 moves into contact with proximally positioned movable handle 1412 when selector lever 1417 is moved to the unlocked position and locks movable handle 1412 in the proximal most position after the gear rack 1421 has been advanced by drivetrain 1418. Also shown on movable handle 1412 is notch 1490, locking protrusion (e.g., projection) 1489 and, when assembled, rod (e.g., bar) 1494 spans the opening in the top of the handle. Shown on gear rack 1421 is protrusion (e.g., raised edge) 1491 and gear rack slot 1493.

When movable handle 1412 is moved to the proximal most position, the frictional force of friction cam (e.g., friction cam 1353 in FIG. 13) and its associated spring on movable handle 1412 will hold movable handle 1412 in the proximal most position. A user/physician can open the jaws by moving the movable handle 1412 distally manually, by forcing the movable handle 1412 distally, or by using the reverse function of the selector lever 1417.

Locking protrusion 1489 will prevent selector cam 1463 and selector lever 1417 from being rotated to the unlocked position if movable handle 1412 is not in the proximal most position. When movable handle 1412 is moved to its proximal most position and selector lever 1417 and selector cam 1463 are turned to the unlocked position, surface (e.g., surface 1788 in FIG. 17B) of selector cam 1463 will move into contact with locking protrusion 1489 and hold or lock movable handle 1412 in the clamped position.

When gear rack 1421 is in a proximal position, such that jaws can be clamped and unclamped, locking cam 1467 will be resting on protrusion 1491 on gear rack 1421 and prevented from moving into notch 1490 on the movable handle 1412. If a user/physician advances the gear rack 1421 distally, protrusion 1491 will also move distally and out of engagement with locking cam 1467. This will allow the distal tip of locking cam 1467 to move into notch 1490 and lock the movable handle 1412 in the proximal position. Locking cam 1467 will stay engaged with notch 1490 until gear rack 1421 is moved proximally and into a position where the clamp/unclamp of the jaws can be performed (e.g., a grasping position of the gear rack 1421) as protrusion 1491 will, at this point, force locking cam 1467 out of notch 1490.

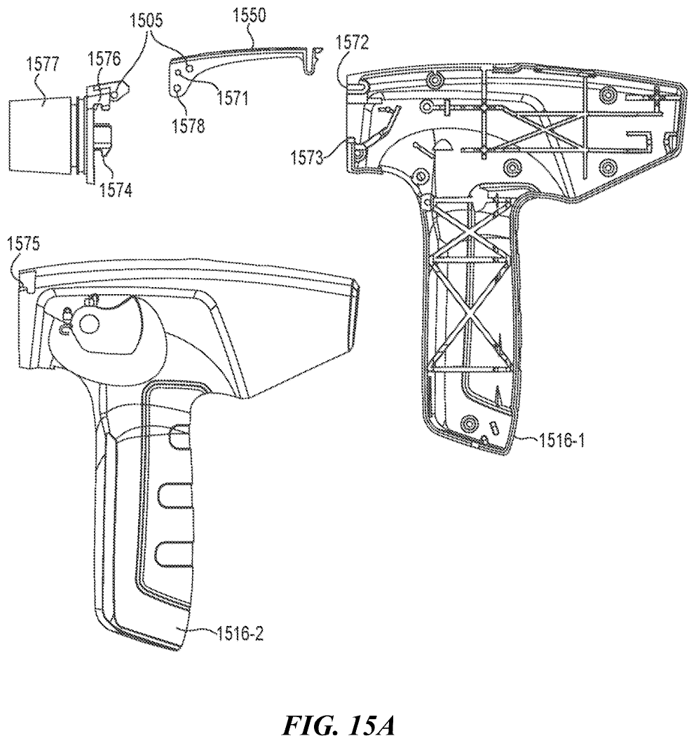

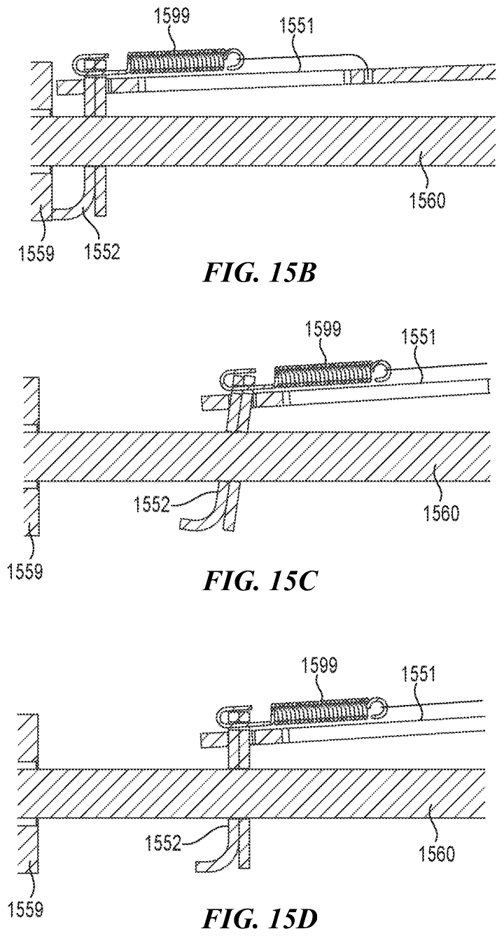

FIGS. 15A, 15B, 15C, and 15D are schematic diagrams of a bailout mechanism for a motorized surgical handle assembly (e.g., motorized surgical handle assembly 1302 in FIG. 13) in accordance with a number of embodiments of the present disclosure. In the event that a surgical stapler, such as apparatus 100 in FIG. 1, malfunctions during use, the user needs a way to open the jaws, so that the jaws are no longer clamping tissue.

In a number of embodiments, a bailout mechanism is provided including a bailout handle 1550 pivotably connected to bailout bar (e.g., bailout bar 1351 in FIG. 13). A cam pin, not shown, extends through cam opening 1571 of bailout handle 1550 and is positioned in cam slot 1572. Bailout bar is rotationally connected to bailout handle 1550 at connection 1578. When fully assembled, distal upper hook 1576 is positioned within proximal upper hook 1575 and distal lower hook 1574 is positioned within proximal lower hook 1573. The distal hooks 1576 and 1574 are positioned on nose cone 1577 and the proximal hooks 1573 and 1575 are positioned on first and/or second handle halves 1516-1 and 1516-2. In some embodiments, there are two upper and two lower hook pairs.

When a user moves the proximal end of bailout handle 1550 up and away from the motorized surgical handle assembly, the cam pin will be forced in a downward direction as cam opening 1571 is positioned distal to the pivot point 1505 for bailout handle 1550. This downward force will put pressure on cam slot 1572 forcing first and second handle halves 1516-1 and 1516-2 in a downward direction. The downward movement of the first and second handle halves 1516-1 and 1516-2 will cause proximal lower hook 1573 to move away from distal lower hook 1574 and proximal upper hook 1575 to move away from distal upper hook 1576. At this point, a proximal casing of the motorized surgical handle assembly including the first and second handle halves 1516-1 and 1516-2 is no longer connected to a distal casing including a radial positioner (e.g., radial positioner 1224 in FIG. 12), an articulation knob (e.g., articulation knob 1222 in FIG. 12), an associated articulation assembly, an outer shaft (e.g., outer shaft 1261 in FIG. 12), and components of a reloadable cartridge assembly (e.g., reloadable cartridge assembly 103 in FIG. 1).

Prior to moving bailout handle 1550, the bailout mechanism is as shown in FIG. 15B. With a reloadable cartridge assembly attached, articulation lock 1559 has been moved proximally against a distal facing protrusion on the distal most toggle plate 1552 and moves toggle plates 1552 into a perpendicular or relatively perpendicular relationship with drive shaft 1560. In this relationship, the toggle plates 1552 do not interfere with the movement of drive shaft 1560. In this embodiment, two toggle plates 1552 are shown. Any number such as 1, 2, 3, 4, or more toggle plates 1552 can be used as long as the distal most toggle plate 1552 has a distal facing protrusion. In some embodiments, the articulation lock 1559 or another component could have a protrusion that would bring a cylindrical toggle plate(s) 1552 into the perpendicular or relatively perpendicular relationship with the drive shaft 1560 and the distal facing protrusion will not be needed.

As the user rotates bailout handle 1550, bailout bar is moved proximally and toggle plates 1552 are moved out of contact with articulation lock 1559. As shown in FIG. 15C, once the toggle plates 1552 are out of contact with articulation lock 1559 and also due to the force of bailout spring 1599, the top of toggle plates 1552 will move proximally causing the toggle plates 1552 to come into contact with and bite on drive shaft 1560. As the bailout handle 1550 is rotated upward, distal outer casing is forced distal relative to the drive shaft 1560 as the bailout handle 1550 is attached to the distal outer casing.

Likely, as the surgical stapler will be in use when the bailout mechanism is used, this distal motion of the distal outer casing will cause drive shaft 1560 and the partial motorized surgical handle assembly to move proximally as the jaws will be clamped on tissue. During this movement, neither the drive shaft 1560 or the gear rack (e.g., gear rack 1424 in FIG. 14) will move relative to the first and second handle halves 1516-1 and 1516-2, a movable handle (e.g., movable handle 1412 in FIG. 14), and/or the drivetrain (e.g., drivetrain 1418 in FIG. 14). After the bailout handle 1550 has been rotated about 90 degrees, the user will rotate the bailout handle 1550 in the opposite direction (e.g., downward toward the motorized surgical handle assembly) and bailout bar will be moved distally.

As shown in FIG. 15D, the force of bailout bar on toggle plates 1552 keep the toggle plates 1552 sufficiently orientated such that they do not bite into drive shaft 1560 but rather slide over it. At this point, the user can again rotate the bailout handle 1550 in an upward direction. In some embodiments described in this paragraph, the toggle plates 1552 are held in a perpendicular or relatively perpendicular relationship with the drive shaft 1560 so that the drive shaft 1560 freely moves through the toggle plates 1552. In these embodiments, the angle of the toggle plates 1552 relative to the drive shaft 1560, taking into account the diameter or the opening and thickness of the toggle plate(s) 1552, needs to be such that the drive shaft 1560 can move without interference from the toggle plates 1552. In some embodiments this is 85° to 95° or 88° to 92°.

In some embodiments, a motorized surgical handle assembly (e.g., motorized surgical handle assembly 1302 in FIG. 3) is controlled by circuit board 1655 as shown in FIGS. 16A and 16B. FIG. 16A shows the top of circuit board 1655 and FIG. 16B shows the bottom. Circuit board 1655 may include a microcontroller or microprocessor for controlling all the functions of the motorized surgical handle assembly, a motor driver, a voltage converter, and, as discussed herein, various switches.

For example, cam (e.g., cam 1357 in FIG. 13) hits (e.g., closes) reload switch 1682 to indicate that a reloadable cartridge assembly (e.g., reloadable cartridge assembly 103 in FIG. 1) is attached to the motorized surgical handle assembly. If no reloadable cartridge assembly is attached to the motorized surgical handle assembly, the motorized surgical handle assembly cannot be electrically activated.

Protrusion (e.g., protrusion 1497 in FIG. 14) on reverse cam 1465 hits reverse switch 1684 to cause the drivetrain (e.g., drivetrain 1418 in FIG. 14) to run in reverse. Protrusion (e.g., protrusion 1785 on selector cam (e.g., selector cam 1763 in FIG. 17) hits switch 1683 to put the motorized surgical handle assembly into a ready-to-fire mode.

When power trigger (e.g., power trigger 1310 in FIG. 13) is pulled, it presses protrusion (e.g., protrusion 1395 in FIG. 13) of an intermediate lever (e.g., intermediate lever 1354 in FIG. 13) into contact with power switch 1681 on circuit board 1655. In some embodiments, power switch 1681 is an open/close switch and the drivetrain receives relatively constant power. In some embodiments, power switch 1681 is a variable switch such as a variable resistor, varistor, potentiometer, or other analog sensor so that the harder power trigger is squeezed, the more power the drivetrain will receive and the faster it will run.

The u-shaped latch (e.g., u-shaped latch 1369 in FIG. 3) interacts with switch 1680 on circuit board 1655. When switch 1680 is closed by the contact of u-shaped latch, power is supplied to the circuit board 1655. When bailout handle (e.g., bailout handle 1550 in FIG. 15) is lifted and u-shaped latch is not in contact with switch 1680 (e.g., the switch is open), no power will flow to the circuit board 1655.

Motorized surgical handle assembly can be powered by a battery, which can be rechargeable or disposable, or by an AC power supply. If a rechargeable battery is used, the battery will be positioned so that it can be either removed or recharged. If a disposable battery is used, the handle will also include a drain so that the battery can be drained prior to disposal. If AC power is used, a power converter can be used to convert 120V or 240V AC, at either 50 or 60 Hz, to 24V, or any other suitable voltage, DC. In some embodiments, the handle will be supplied with a power cord that will plug in to the power converter.

This power can be supplied to the circuit board 1655. In some embodiments, the drivetrain is provided with 12V for the forward movement and 24V for the reverse. In some embodiments, the microcontroller may control many features of the motorized surgical handle assembly. For example, no power will be supplied to the drivetrain if no reloadable cartridge assembly is attached to the motorized surgical handle assembly.

As is known in the art, many reloadable cartridge assemblies have a blade lock that is engaged after a reloadable cartridge assembly has been used so that the reloadable cartridge assembly cannot be accidentally used again. In some embodiments, the control system of the microcontroller has a limit such as 0.2 to 0.3 amps for the first 5 to 10 mm of travel of the gear rack (e.g., gear rack 1421 in FIG. 14). In some embodiments, the control system of the microcontroller has a limit such as 0.25 amps for the first 5 to 10 mm of travel. In the event that the blade lock would be engaged, the current to the motor would spike above this limit and the drivetrain would be stopped. In some embodiments, for the remaining stroke length, the limit can be 0.75 to 1.25 amps. In some embodiments, for the remaining stroke length, the limit can be 1.0 amps. This limit is set high enough for a surgical stapler, such as apparatus 100 in FIG. 1, to safely staple and cut through tissue, but when the surgical stapler reaches the end of the firing stroke, the limit will be hit, and the drivetrain will be stopped. At this time, the user can put the surgical handle assembly into reverse to retract the drive shaft.

FIGS. 17A, 17B, and 17C show various views of selector cam 1763. FIG. 17A is a top view. FIG. 17B is a view from the side opposite the selector lever (e.g., selector lever 1417 in FIG. 14). FIG. 17C is from the side that faces the selector lever.

Shown is hole 1796 that houses pin (e.g., pin 1464 in FIG. 14) so that selector cam 1763 is non-rotationally fixed to the selector lever. Also shown is protrusion 1785. When selector lever is moved to the unlocked position, protrusion 1785 closes switch (e.g., switch 1683 in FIG. 6B) on circuit board (e.g., circuit board 1655 in FIGS. 6A and 6B) putting the motorized surgical handle assembly in the ready to fire position.

Shown in FIG. 17C are surfaces 1792, 1786, 1787 of selector cam 1763. As discussed with regard to FIG. 14, when assembled, spring (e.g., spring 1466 in FIG. 4) pushes reverse cam (e.g., reverse cam 1465 in FIG. 14) in a downward clockwise direction when situated as in FIG. 14. When the selector lever is in the locked position, surface 1786 rests against the reverse cam and selector cam 1763 and selector lever are in a stable position.

When selector lever is rotated to the unlocked position, selector cam 1763 rotates such that surface 1792 is positioned against the reverse cam. When the selector lever is in the unlocked position, surface 1792 rests against the reverse cam and selector cam 1763 and selector lever are in a stable position. The transition from surface 1786 to 1792 is not sufficient to cause reverse cam protrusion (e.g., protrusion 1497 in FIG. 14) to hit reverse switch (e.g., reverse switch 1684 in FIG. 6B) on the circuit board.

When the selector lever is rotated to the reverse position, selector cam 1763 rotates such that surface or tip 1787 is positioned against the reverse cam. This causes the reverse cam protrusion to hit the reverse switch (e.g., reverse switch 1684 in FIG. 6B) on the circuit board but it is not a stable position and the spring will force the selector lever back to the locked position after the physician/user releases the selector lever.

Also shown is concave locking opening 1713. When the movable handle is in a proximal position proximate to the non-movable handle and subsequently the selector lever and the selector cam 1763 are rotated to the unlocked position, locking protrusion (e.g., locking protrusion 1489 in FIG. 14) will be positioned in concave locking opening 1713 and will prevent the movable handle from opening (e.g., moving distally).

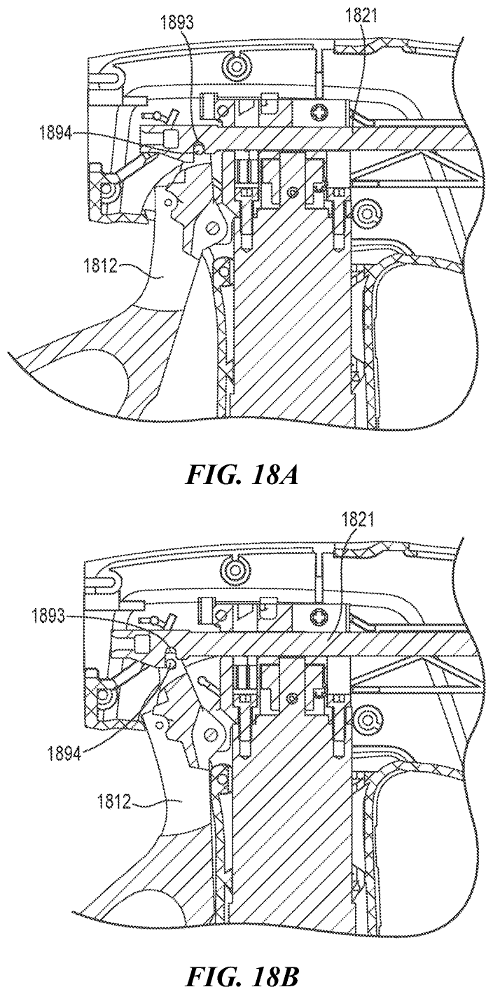

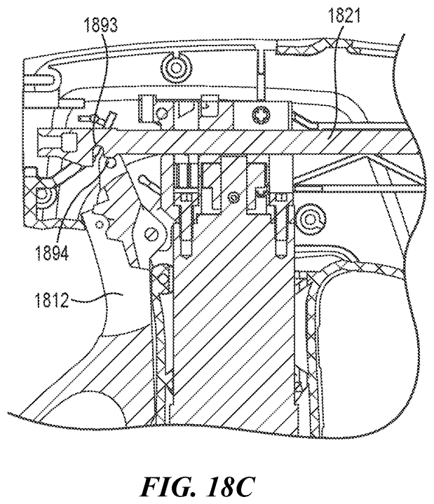

FIGS. 18A, 18B, and 18C show an example of the clamp and unclamp mechanism for a motorized surgical handle assembly (e.g., motorized surgical handle assembly 1302 in FIG. 13) in accordance with a number of embodiments of the present disclosure. FIG. 18A shows a portion of the motorized surgical handle assembly with gear rack 1821 in the clamping position. In the clamping position, rod 1894 is positioned within gear rack slot 1893, movable handle 1812 is in a distal position, and the jaws are in the open or unclamped position.

Gear rack slot 1893 has a general shape of an upside-down “U”, with the distal leg of the “U” being longer than the proximal leg of the “U”. When a user/physician squeezes movable handle 1812 and moves it to the proximal position, as rod 1894 is positioned above the movable handle 1812 pivot point, the rod 1894 will move in a distal direction. As rod 1894 is positioned in gear rack slot 1893, gear rack 1821 will be moved distally when movable handle 1812 is squeezed. This distal movement of gear rack 1821 will cause the jaws to close or clamp. In some embodiments, this distal movement causes an I-beam to move into contact with the two elongated members (e.g., elongated members 107 and 109 in FIG. 1) and causes them to move together.

In this position shown in FIG. 18B, rod 1894 is positioned beneath the shorter proximal leg of gear rack slot 1893 but still positioned against the longer, distal leg of gear rack slot 1893. At this stage, the user/physician can either move movable handle 1812 back to the distal position which will result in gear rack 1821 moving proximally and unclamping or opening the jaws, either manually or using the reverse setting of the selector lever or turn the selector lever to unlock and begin the firing (e.g., staple delivery) process.

FIG. 18C illustrates the position of gear rack 1821 after the user/physician has begun the firing process. As rod 1894 was positioned beneath the end of the shorter, proximal leg of gear rack slot 1893, it will not interfere with any distal movement of gear rack 1821. FIG. 18C shows that gear rack 1821 has moved distal of the clamping position shown in FIG. 18B and rod 1894 has moved out of gear rack slot 1893 and is positioned proximal to gear rack slot 1893.

As described above, when a user/physician uses the reverse position of the selector lever after delivering staples, the gear rack 1821 is returned to the position shown in FIG. 18B. At this point, the user/physician can either unclamp the jaws by manually moving movable handle 1812 distal or by turning selector lever to reverse and allowing the electric motor to open the jaws. FIG. 18A is the position that the control system will put the motorized surgical handle assembly in upon powering up of the motorized surgical handle assembly and attaching a reloadable cartridge assembly, provided that the reloadable cartridge assembly is not attached to the motorized surgical handle assembly at startup.

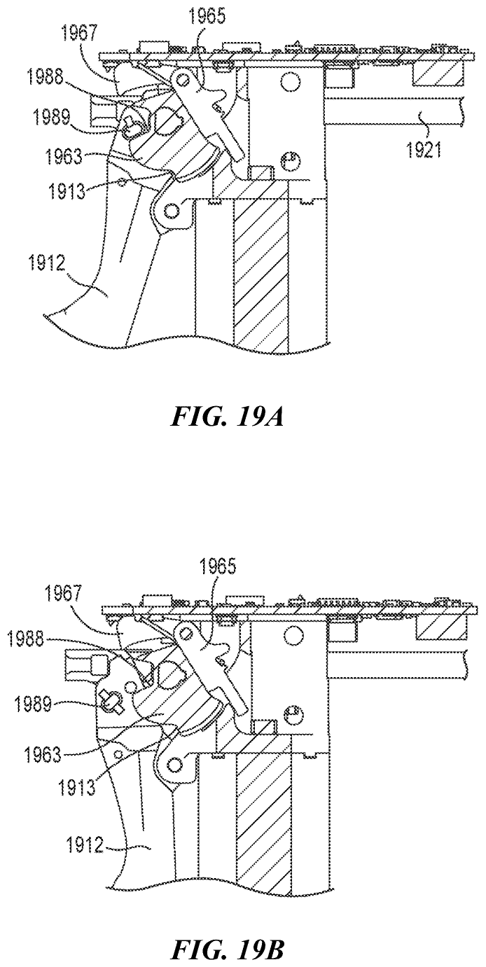

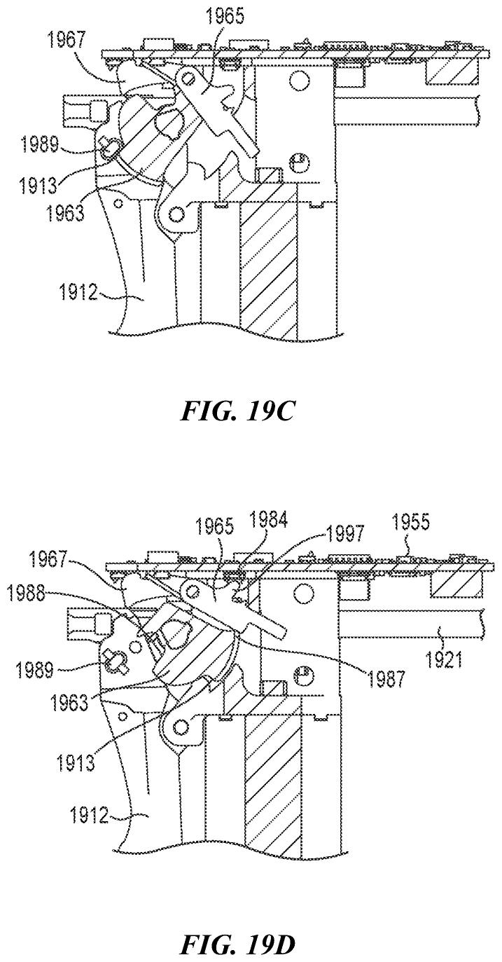

FIGS. 19A, 19B, 19C, and 19D show an example of a selector cam 1963 in various settings for a motorized surgical handle assembly (e.g., motorized surgical handle assembly 1302 in FIG. 13) in accordance with a number of embodiments of the present disclosure. FIG. 19A shows a portion of the motorized surgical handle assembly with the movable handle 1912 in a distal position and a selector lever (e.g., selector lever 1417 in FIG. 14) (not shown) in the locked position. In this position, locking protrusion 1989 is positioned against surface 1988 and prevents the selector cam 1963 (and the selector lever) from being rotated to the unlocked position or, in this figure, rotated clockwise.

In FIG. 19B, movable handle 1912 has been moved to the proximal position and locking protrusion 1989 has moved out of contact with surface 1988 and/or selector cam 1963. Now, selector cam 1963 (and the selector lever) can be rotated to the unlocked position or, in this figure, rotated clockwise.

In FIG. 19C, the selector cam 1963 (and selector lever) has been rotated to the unlocked position to put the motorized surgical handle assembly in the ready to fire position. In this position, locking protrusion 1989 is now against concave locking opening 1913 of selector cam 1963. In this position, selector cam 1963 locks movable handle 1912 in the proximal position.

FIG. 19D shows the selector cam 1963 (and selector lever) in the reverse position. In this position, locking protrusion 1989 is no longer positioned against concave locking opening 1913 and the selector cam 1963 is no longer locking the movable handle 1912 in the proximal position. Here, if gear rack 1921 is in a position distal to the clamping position, locking cam 1967 will be positioned within notch (e.g., notch 1490 in FIG. 14) of movable handle 1912 and will be acting to lock the movable handle in the proximal position. If gear rack 1921 is in the clamping position, friction cam (e.g., friction cam 1353 in FIG. 13) will be positioned against movable handle 1912 and, through friction, holding the movable handle in the proximal position. Shown is reverse cam 1965 moved to an upward position by tip 1987 with protrusion 1997 in a position to close reverse switch 1984 of circuit board 1955.

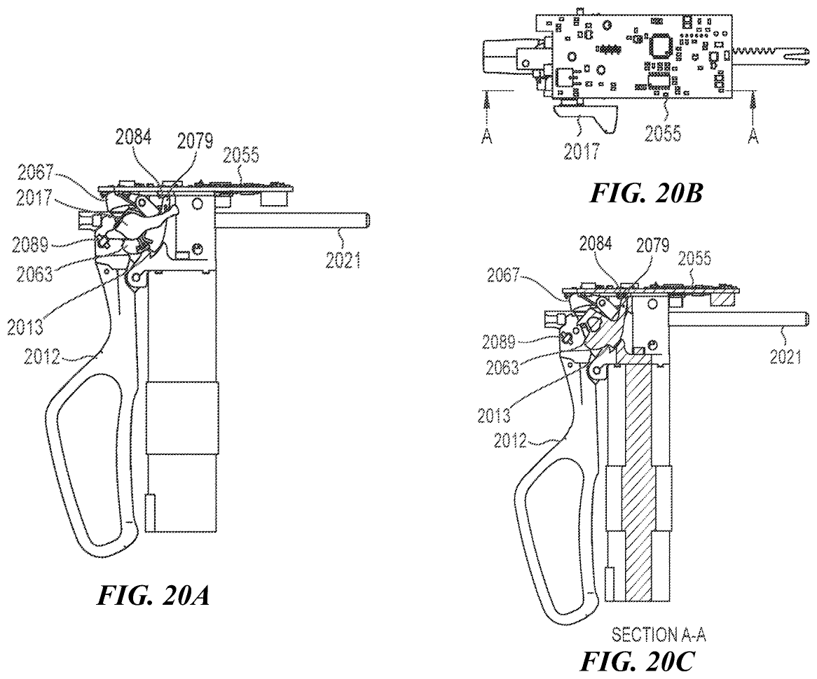

FIGS. 20A, 20B, and 20C show the selector cam 2063 and selector lever 2017 in the reverse position. In this position, locking protrusion 2089 is no longer positioned against concave locking opening 2013 and the selector cam 2063 is no longer locking the movable handle 2012 in the proximal position. Here, if gear rack 2021 is in a position distal to the clamping position, locking cam 2067 will be positioned within notch (e.g., notch 1490 in FIG. 14) of movable handle 2012 and will be acting to lock the movable handle 2012 in the proximal position. If gear rack 2021 is in the clamping position, friction cam (e.g., friction cam 1353 in FIG. 13) will be positioned against movable handle 2012 and, through friction, holding the movable handle 2012 in the proximal position. Shown is selector cam 2063 moved to an upward position with protrusion 2079 in a position to close reverse switch 2084 of circuit board 2055. Accordingly, as shown in FIGS. 20A, 20B, and 20C, the selector cam 2063 can directly trigger the reverse switch 2084 of the circuit board 2055 without the reverse cam (e.g., reverse cam 1965 in FIG. 19D).

As discussed herein, the circuit board 2055 of the motorized surgical handle assembly may include a microcontroller or microprocessor to control and/or automate various aspects of the motorized surgical handle assembly. For example, in some embodiments the microcontroller runs through a series of checks upon start up when the device is turned on or, in some embodiments, when it is plugged in. If no reloadable cartridge assembly (e.g., reloadable cartridge assembly 103 in FIG. 1) is attached, the microcontroller will reverse the drivetrain (e.g., drivetrain 1418 in FIG. 14) until the gear rack 2021 is in the proximal-most position and the retraction spring (e.g., retraction spring 1356 in FIG. 3) is compressed. In some embodiments this is called the home position. At this point, upon detecting a current spike, the drivetrain will rotate the driving gear to move the gear rack 2021 distally a short distance by the unloading of the retraction spring. In some embodiments, this is called the loading position.

If, upon start up, a reloadable cartridge assembly is detected, no action will be taken. In some embodiments where the motorized surgical handle assembly is powered up with no reloadable cartridge assembly attached and where the control system moves the gear rack 2021 to the loading position, upon a reloadable cartridge assembly being attached, the control system will rotate the driving gear (e.g., driving gear 1043 in FIGS. 10A and 10B) a short distance, in some embodiments about 70°, while not moving driven gear (e.g., driven gear 1044 in FIGS. 10A and 10B) or gear rack 2021. This is the clamping or grasping position. This movement of the driving gear causes bosses (e.g., bosses 1045-1 and 1045-2 in FIG. 10B) to be positioned relative to bosses (e.g., bosses 1045-3 and 1045-4 in FIG. 10A) of the driven gear to allow space for the clamping motion, which moves the gear rack 2021 distally without interference from the bosses.

In some embodiments, an encoder is used with the drivetrain. The microcontroller can receive information from the encoder and will use the encoder information, for example, in setting the current limits for the distal movement of the gear rack 2021. In some examples, when the motorized surgical handle assembly is put into reverse, the reverse motion will continue until the gear rack 2021 is in a position wherein the movable handle 2012 can be used to open and close, unclamp and clamp, the jaws.

If the selector lever 2017 is again turned to the reverse setting, the gear rack 2021 will be moved to the proximal most position, whereupon it may be moved distally a small distance as described herein and the jaws will automatically unclamp. However, if while the gear rack 2021 is being reversed a physician/user moves the selector lever 2017 to the reverse or unlocked position or presses the power trigger (e.g., power trigger 1310 in FIG. 13), the reverse motion will stop. At this point the physician/user can again move the selector lever 2017 to reverse to continue the reverse action or move the selector lever to the unlocked position and move the gear rack 2021 distally.

In some embodiments, the microcontroller determines when the I-beam of the reloadable cartridge assembly is at its end of stroke by sensing a current spike in the drivetrain. When the current gets above a set point, the microcontroller will stop the drivetrain. In some embodiments, the microcontroller may rely on sudden current drops and/or sudden rises in current to indicate that there may be a problem somewhere in the surgical stapler. For example, a sudden drop in current may indicate a breakage somewhere in the surgical stapler and a sudden rise in current may indicate the end of a stroke, an obstruction, or tissue that is too thick or tough for the surgical stapler.

In some embodiments, artificial intelligence of the microcontroller may be used to capture the various signals from the surgical stapler and analyze the signals to determine if there are problems with the surgical stapler or if there are ways to improve the performance for the physician/user. In some embodiments, the encoder is used to inform the microprocessor of the relative position of the gear rack 2021. In this embodiment, the microprocessor could, for example, zero out the location indicator for the gear rack 2021 when the gear rack 2021 has compressed the retraction spring. In this example the microprocessor would then be able to determine when to switch from the low amp setting to the high amp setting and would be able to determine when the gear rack 2021 is properly positioned to allow for the movable handle 2012 to clamp and unclamp the jaws.

In some embodiments, in addition to or as an alternative to actuating a retract button (e.g., the retract button 114 in FIG. 1) and/or a selector lever (e.g., the selector lever 1317 of FIG. 13), rotation of a movable handle (e.g., the movable handle 1312 of FIG. 13) can be used as an input for triggering a drivetrain (e.g., the drivetrain 1318 in FIG. 13) to move or actuate a drive assembly (e.g., a rotary-to-linear transmission assembly, such as a lead screw, a ball screw, or a gear rack (e.g., the gear rack 1321 of FIG. 13); a drive shaft (e.g., the drive shaft 1260, 1360, and/or 1560 of FIGS. 12, 13, and/or 15, respectively); and/or a blade shaft) in a distal or proximal direction. For example, a user can move the movable handle in a distal (or unclamping) direction to trigger (a) electrically assisted retraction (e.g., proximal movement) of the drive assembly and/or (b) electrically assisted unclamping of jaws (e.g., the first and second elongated members 107, 109 of FIG. 1) of a reloadable cartridge assembly coupled to the motorized surgical handle assembly apparatus.

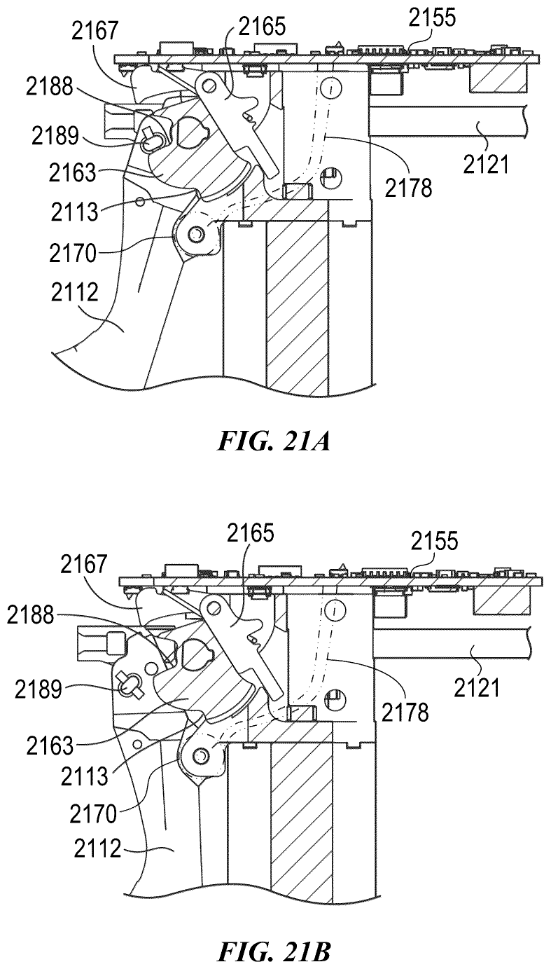

As discussed herein, FIGS. 21A-23 illustrate various sensor types and arrangements that are usable to obtain measurements corresponding to a position and/or orientation of the movable handle. In some embodiments, the drivetrain, operated by a controller (e.g., a microprocessor or microcontroller), provides electrically-assisted retraction and/or unclamping in response to a sensor output indicating that the movable handle has moved away from the non-movable handle by a threshold amount. The threshold amount can be between 1-30 degrees, 1-20 degrees, 1-10 degrees, 1-5 degrees, etc. (e.g., about 2 degrees, 3 degrees, 5 degrees, 10 degrees, 15 degrees, 20 degrees, 25 degrees). In some embodiments, the controller also determines the direction of the movement of the movable handle based on the sensor output, and moves a drive assembly (e.g., the gear rack 1321 of FIG. 13) in the proximal direction when the movable handle is moving in the distal direction (or the unclamping direction).

FIGS. 21A and 21B show an example of a sensor 2170 usable with a movable handle 2112 for a motorized surgical handle assembly apparatus (e.g., the motorized surgical handle assembly 1302 in FIG. 13) in accordance with a number of embodiments of the present disclosure. More specifically, FIG. 21A illustrates the movable handle 2112 in a relatively distal position and FIG. 21B illustrates the movable handle 2112 in a relatively proximal position. As shown, the motorized surgical handle assembly can include a reverse cam 2165, a locking cam 2167, a selector cam 2163, and a gear rack 2121. The selector cam 2163 can include a concave locking opening 2113 and a surface 2188. The movable handle 2112 can include a protrusion 2189.

The sensor 2170 can include a rotational potentiometer, a linear potentiometer, or other suitable sensor coupled to the movable handle 2112. Therefore, as the movable handle 2112 rotates (e.g., manually by a user), the sensor 2170 can measure changes in the position and/or orientation of the movable handle 2112 via analog or digital means. For example, in the illustrated embodiment, the sensor 2170 comprises a rotational potentiometer positioned at or near the pivot point of the movable handle 2112 such that the rotational potentiometer is generally coaxial with the axis of rotation of the movable handle 2112. The sensor 2170 can be electrically coupled to a circuit board 2155 via one or more wires 2178. In some embodiments, the sensor 2170 can continuously measure the angle of the movable handle 2112 relative to a reference point (e.g., a proximal-most orientation of the movable handle 2112 (as shown in FIG. 21B) or a distal-most orientation of the movable handle 2112 (as shown in FIG. 21A)). In these and other embodiments, the sensor 2170 can continuously measure the angle of the movable handle 2012 relative to a reference point within (e.g., only within) a region of interest, such as between (i) a proximal-most orientation of the movable handle 2112 and (ii) an orientation between the proximal-most orientation and a distal-most orientation of the movable handle 2112. A microcontroller or microprocessor of the circuit board 2155 can receive angle measurements from the sensor 2170 over time, compare them against a threshold value, and actuate the drivetrain (not shown) to drive a drive assembly (e.g., including the gear rack 2121) in a proximal direction when the angle measurements indicate distal movement of the movable handle 2112 equal to or greater than the threshold value. Driving the drive assembly can enable electrically assisted retraction of a drive shaft (not shown) and/or electrically assisted unclamping of the jaws (not shown).

In some embodiments, the microcontroller or microprocessor of the circuit board 2155 can actuate the drivetrain (not shown) continuously and/or dynamically in response to the received measurements (e.g., rather than actuating the drivetrain when or only when the received measurements exceed a threshold value). In other words, actuation of the drivetrain can be directly correlated with the received measurements so that the drivetrain can drive the drive assembly in a proximal or distal direction in real-time, by an amount corresponding to the received measurements, and/or by a speed or rate corresponding to the received measurements. For example, the microcontroller or microprocessor can actuate the drivetrain so that the angle between the jaws (e.g., the first and second elongated members 107, 109 of FIG. 1) of the reloadable cartridge assembly increases incrementally as the movable handle 2112 is moved distally, and decreases incrementally as the movable handle 2112 is moved proximally. In some embodiments, the angle between the jaws changes linearly with the position of the movable handle 2112 (e.g., the jaws are opened halfway when the movable handle 2112 is moved halfway between its proximal-most and distal-most positions). In other embodiments, the relationship between the angle between the jaws and the position of the movable handle 2112 is non-linear.

In another example, the microcontroller or microprocessor can actuate the drivetrain so that the angle between the jaws changes at a rate corresponding to the rate of movement of the movable handle 2112 determined based on the received measurements. The sensor 2170 can measure the angle of the movable handle 2112 relative to a reference point on a millisecond or other suitable time scale (e.g., can take measurements every 1 ms, 5 ms, 10 ms, 50 ms, 100 ms, etc.). The reference point can be a fixed point (e.g., a proximal-most position/orientation of the movable handle, a distalmost position/orientation of the movable handle, a midpoint position/orientation of the movable handle between the proximal-most position/orientation and the distalmost position/orientation, a position or orientation of another reference point on the surgical handle assembly apparatus). Alternatively, the reference point can be one or more previous positions and/or orientations of the movable handle (e.g., corresponding to one or more previous measurements of the position/orientation of the movable handle) and can therefore change as the movable handle is actuated. By taking measurements at a high frequency, the sensor 2170 can allow the drivetrain to clamp or unclamp the jaws at a rate that mirrors, tracks, is proportional to, and/or otherwise corresponds to the rate of the rotation of the movable handle 2112. Accordingly, the microcontroller or microprocessor can actuate the drivetrain to enable controlled, electrically-assisted unclamping and grasping functionality in real-time based on the received sensor measurements.

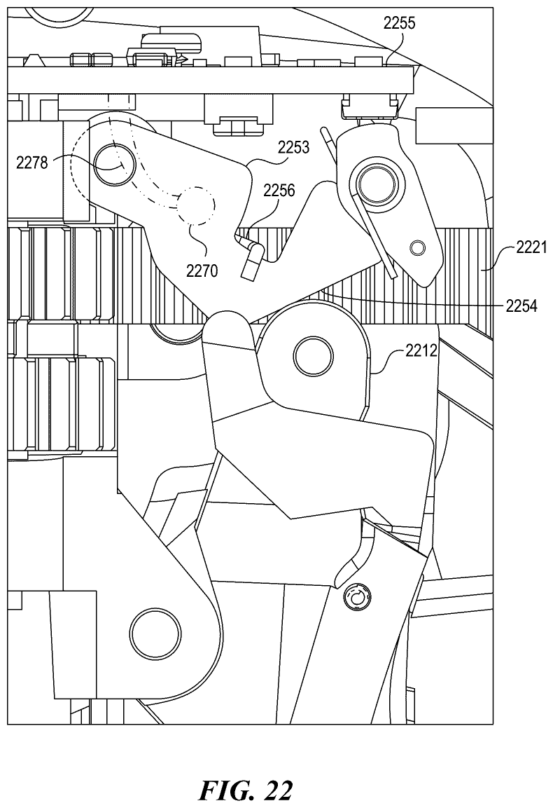

FIG. 22 shows an example of a sensor 2270 positioned on a friction cam 2253 (e.g., the friction cam 1353 of FIG. 13) for a motorized surgical handle assembly (e.g., the motorized surgical handle assembly 1302 in FIG. 13) in accordance with a number of embodiments of the present disclosure. In the illustrated embodiment, the top portion of a movable handle 2212 abuts against a distal surface 2254 of the friction cam 2253. A biasing member 2256 (e.g., a spring) can be positioned to apply a biasing force against the friction cam 2253 such that the friction cam 2253 remains in contact with and rotates with the movable handle 2212. In other words, the biasing member 2256 can be used to maintain contact of the distal surface 2254 of the friction cam 2253 with the movable handle 2212 such that rotation/movement of the movable handle 2212 can cause corresponding rotation/movement of the friction cam 2253 about a pivot point of the friction cam 2253.

The sensor 2270 can include a rotational potentiometer, a linear potentiometer, or another suitable sensor coupled to the friction cam 2253. For example, in the illustrated embodiment, the sensor 2270 comprises a linear potentiometer coupled to a portion of the friction cam 2253 that is positioned away from the pivot point of the friction cam 2253. The sensor 2270 can be electrically coupled to a circuit board 2255 via one or more wires 2278. In some embodiments, the sensor 2270 can continuously measure the position of the portion of the friction cam 2253. Because (i) the distance between the pivot point of the friction cam 2253 and the sensor 2270 and (ii) the rotational relationship between the friction cam 2253 and the movable handle 2212 are known, a microcontroller or microprocessor of the circuit board 2255 can use an output of the sensor 2270 (indicating the position of the portion of the friction cam 2253) as a proxy for an amount of rotation of the movable handle 2212. Therefore, the microcontroller or microprocessor of the circuit board 2255 can compare the sensor output against a proxy threshold value, and actuate the drivetrain (not shown) to move a drive assembly (e.g., including the gear rack 2221) in a proximal direction or distal direction when sensor output indicates distal movement/rotation of the movable handle 2212 equal to or greater than the proxy threshold value.

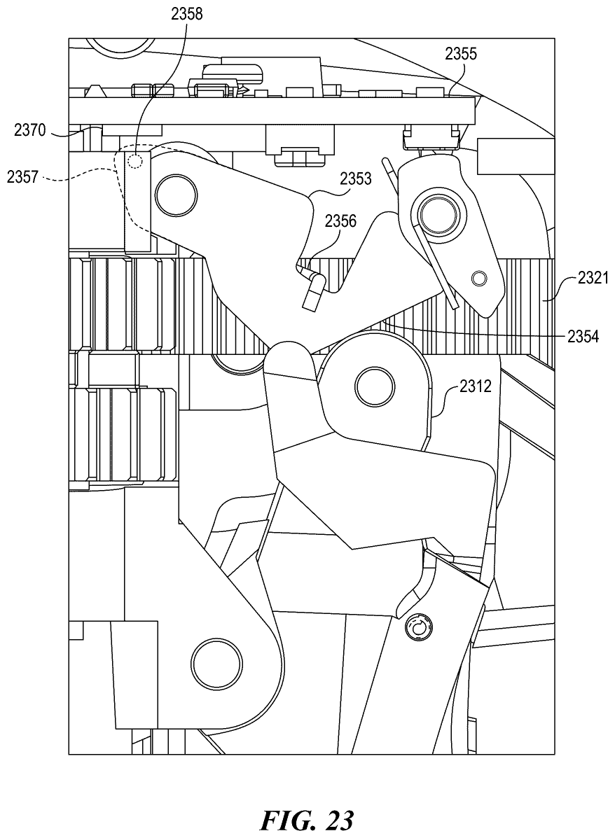

FIG. 23 shows an example of a sensor 2370 on a circuit board 2355 for a motorized surgical handle assembly (e.g., the motorized surgical handle assembly 1302 in FIG. 13) in accordance with a number of embodiments of the present disclosure. In the illustrated embodiment, a top portion of a movable handle 2312 abuts against a distal surface 2354 of a friction cam 2353. A biasing member 2356 (e.g., a spring) can be positioned to apply a biasing force against the friction cam 2353 such that the friction cam 2353 remains in contact with and rotates with the movable handle 2312. In other words, the biasing member 2356 can be used to maintain contact of the distal surface 2354 of the friction cam 2353 with the movable handle 2312 such that rotation/movement of the movable handle 2312 causes corresponding rotation/movement of the friction cam 2353 about a pivot point of the friction cam 2353.

The sensor 2370 can include a binary switch, a non-contact sensor (e.g., a Hall effect sensor), or another suitable sensor positioned to measure a position and/or orientation of the friction cam 2353. For example, in the illustrated embodiment, the sensor 2370 comprises a binary switch attached to a bottom surface of the circuit board 2355. The sensor 2370 can be electrically coupled to a circuit board 2355 via one or more wires 2378. The binary switch can be actuated upon contact with (and/or depression by) a proximal portion 2357 of the friction cam 2353. As discussed above, because the friction cam 2353 can rotate with the movable handle 2312, actuation of the binary switch can serve as an indication that the movable handle 2312 has moved distally by a threshold amount relative to a proximal (or proximal-most) orientation of the movable handle 2312. Therefore, a microcontroller or microprocessor of the circuit board 2355 can, upon actuation of the binary switch, operate the drivetrain (not shown) to provide electrically assisted retraction and/or unclamping. The position of the sensor 2370 and/or the size and shape of the proximal portion 2357 of the friction cam 2353 can be selected such that actuation of the sensor 2370 accurately corresponds to distal movement of the movable handle 2312 by at least the desired threshold amount.

In some embodiments, the proximal portion 2357 of the friction cam 2353 can include a magnet 2358 and the sensor 2370 can include a Hall effect sensor. In operation, the sensor 2370 can measure the position and/or orientation of the magnet 2358. Because (i) the distance between the pivot point of the friction cam 2353 and the sensor 2370, (ii) the distance between the pivot point of the friction cam 2353 and the magnet 2358, and (iii) the rotational relationship between the friction cam 2353 and the movable handle 2312 are known, the microcontroller or microprocessor of the circuit board 2355 can use an output of the sensor 2370 (indicating the position of the magnet 2358) as a proxy for an amount of rotation of the movable handle 2312. Therefore, the microcontroller or microprocessor of the circuit board 2355 can compare the sensor output against a proxy threshold value, and actuate the drivetrain (not shown) to move a drive assembly (e.g., including the gear rack 2321) (a) in a proximal direction when sensor output indicates distal movement/rotation of the movable handle 2312 equal to or greater than the proxy threshold value.

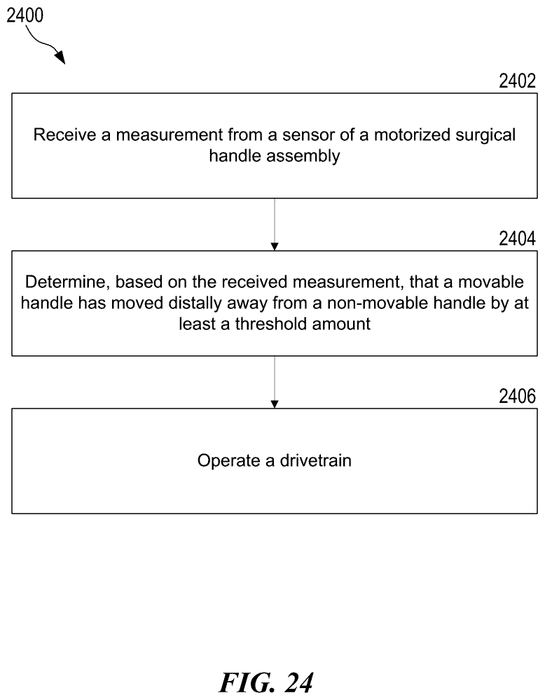

FIG. 24 is a flowchart illustrating a method 2400 for operating a motorized surgical handle assembly apparatus in accordance with a number of embodiments of the present disclosure. While the steps of the method 2400 are described below in a particular order, one or more of the steps can be performed in a different order or omitted, and the method 2400 can include additional and/or alternative steps. Additionally, although the method 2400 may be described below with reference to the embodiments of the present technology described herein, the method 2400 can be performed with other embodiments of the present technology.

The method 2400 begins at block 2402 by receiving a measurement from a sensor (e.g., the sensors 2170, 2270, 2370 of FIGS. 21A, 22, and 23, respectively) of a motorized surgical handle assembly apparatus. The measurement can correspond to at least one of a position or orientation of a movable handle of the motorized surgical handle assembly apparatus. In some embodiments, the sensor comprises a rotational potentiometer coupled to the movable handle, and the measurement comprises an angle of rotation of the movable handle.

In some embodiments, the sensor comprises a linear potentiometer coupled to a friction cam movably coupled to the non-movable handle. The friction cam can be biased to move with the movable handle, and the measurement can comprise a change in position of the friction cam. In some embodiments, the sensor comprises a rotational potentiometer coupled to the friction cam, and the measurement can comprise an angle of rotation of the friction cam. In some embodiments, the sensor comprises a binary switch or a non-contact sensor (e.g., a Hall effect sensor).

At block 2404, the method 2400 continues by determining, based on the received measurement, that the movable handle has moved (e.g., distally) away from a non-movable handle of the motorized surgical handle assembly apparatus by at least a threshold amount. In embodiments in which the sensor is not directly attached to the movable handle (e.g., in embodiments in which the sensor is attached to the friction cam or a circuit board), the sensor output can indicate that the friction cam (or other component) has moved by an amount corresponding to movement of the movable handle equal to or greater than the threshold amount. The threshold amount can be between 1-30 degrees, 1-20 degrees, 1-10 degrees, 1-5 degrees, etc. (e.g., about 2 degrees, 3 degrees, 5 degrees, 10 degrees, 15 degrees, 20 degrees, 25 degrees), such as relative to a proximal (e.g., proximal-most) orientation of the movable handle.

At block 2406, the method 2400 continues by operating a drivetrain of the motorized surgical handle assembly apparatus. Operating the drivetrain can comprise moving a drive assembly (e.g., driving a gear rack) of the motorized surgical handle assembly apparatus in a distal or proximal direction. Operating the drivetrain can comprise clamping or unclamping jaws (e.g., the first and second elongate members 107, 109 of FIG. 1) of a reloadable cartridge assembly coupled to the motorized surgical handle assembly apparatus. Operating the drivetrain can comprise moving the movable handle distally away from the non-movable handle.

Although discussed above in the context of clamping or unclamping jaws of a reloadable cartridge assembly coupled to a motorized surgical handle assembly apparatus, the present technology is not so limited. For example, a drivetrain of a surgical handle assembly apparatus can be actuated based on sensor output in accordance with the discussion above to provide electrically-assisted actuation of an end effector of a type other than linear surgical staplers and corresponding jaws. Specific examples of other types of end effectors that can be actuated using a drivetrain based on sensor output relating to positions/orientations of a movable handle of a motorized surgical handle assembly apparatus include circular staplers, grippers, scissors, forceps, and drills, among other suitable end effectors.

Referring to FIGS. 1-24 together, in some embodiments, it is important for the motor to immediately stop when the end of stroke is detected as continued movement of the drivetrain after the end of stroke is reached could result in damage to the reloadable cartridge assembly. In some embodiments, the software or control system of the microcontroller can use the motor speed and motor drive current to detect obstructions or potential obstructions in the path of the surgical stapler. In this embodiment, the control system can determine the difference between a hard tissue that the surgical stapler can progress through and an obstruction such as a piece of surgical equipment or the end of the reloadable cartridge assembly fire stroke and control the motor as appropriate.

In some embodiments, the control system can be preprogrammed with the various lengths of reloadable cartridge assemblies that may be used with the surgical handle. In this embodiment, the control system can be programmed to more quickly cut power if an obstacle is detected at any of these lengths in an effort to prevent damage to the reloadable cartridge assembly.

In some embodiments, a visible light, such as a LED, or audible system may be used to inform the user/physician of the status of the motorized surgical handle assembly. In this embodiment, for example, the light or LED could be green when everything is fine, flashing green when in the ready to fire position, yellow if a possible obstruction is detected, and red if the drivetrain is stopped for an obstruction including the end of firing stroke or if some other problem is detected. In some embodiments, the light can be an LED positioned beneath a transparent tradename on the top of the motorized surgical handle assembly. In some examples, the motorized surgical handle assembly could emit sound, such as, beeps which can change in pitch or frequency when there is a problem.

In a number of embodiments, a method of using a surgical stapler is provided. When positioning the surgical stapler for use, either in open surgery or through an instrument such as a trocar, the clinician may need to rotate and/or articulate the elongated members to position them properly. As the surgical stapler is used to clamp, staple, and cut tissue, in some instances the clinician needs to clamp and unclamp the elongated members to ensure that they are properly positioned. Once the elongated members are in position, the clinician can clamp the elongated members and deliver the staples. The cutter on the I-beam can cut the tissue as the staples are being delivered.

Some of the embodiments described herein have a motorized surgical handle assembly with a reloadable staple or cartridge assembly. The embodiments herein are equally applicable to a staple system where the reloadable cartridge is just a staple cartridge that is loaded into one of the elongated members.

In some embodiments discussed herein such as, for example, FIG. 3B, various components are shown as separate pieces. Multiple components can be formed as a single piece and still fall within the scope of the inventions discussed herein. In some embodiments discussed herein, the motorized surgical handle assembly may also have an on/off switch. In some embodiments discussed herein, the motorized surgical handle assembly will power up immediately upon being plugged in to a power source.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled. The terms proximal and distal, as used herein, are from the perspective of the clinician or other user of the device. The terms left and right, as used herein, are from the perspective of a clinician or other user holding apparatus (e.g., apparatus 100 in FIG. 1) in an upright position with non-movable handle (e.g., non-movable handle 1116 in FIG. 11) on the bottom.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

I/We claim:

1. A motorized surgical handle assembly apparatus, comprising:
a non-movable handle;
a movable handle movably coupled to the non-movable handle;
a drive assembly;
a drivetrain operably coupled to the movable handle and configured to move the drive assembly;
a sensor configured to obtain a measurement corresponding to at least one of a position or orientation of the movable handle; and
a controller operably coupled to the drivetrain and the sensor, wherein the controller is configured to:
determine, based at least in part on the measurement, that the movable handle has moved away from the non-movable handle by at least a threshold amount, and
in response to determining that the movable handle has moved away from the non-movable handle by at least the threshold amount, operate the drivetrain to retract the drive assembly.

2. The motorized surgical handle assembly apparatus of claim 1, wherein, in response to determining that the movable handle has moved away from the non-movable handle by at least the threshold amount, the controller is configured to operate the drivetrain to retract the drive assembly such that the movable handle is moved away from the non-movable handle.

3. The motorized surgical handle assembly apparatus of claim 1, wherein, in response to determining that the movable handle has moved away from the non-movable handle by at least the threshold amount, the controller is configured to operate the drivetrain to retract the drive assembly such that jaws of a reloadable cartridge assembly coupled to the motorized surgical handle assembly apparatus are unclamped.

4. The motorized surgical handle assembly apparatus of claim 1, wherein the sensor comprises a rotational potentiometer coupled to the movable handle such that the rotational potentiometer is coaxial with an axis of rotation of the movable handle, and wherein the measurement indicates an angle of rotation of the movable handle.

5. The motorized surgical handle assembly apparatus of claim 1, further comprising:
a friction cam movably coupled to the non-movable handle; and
a spring configured to bias the friction cam toward the movable handle such that movement of the movable handle moves the friction cam by a corresponding amount.

6. The motorized surgical handle assembly apparatus of claim 5, wherein the sensor comprises a rotational potentiometer coupled to the friction cam such that the rotational potentiometer is coaxial with an axis of rotation of the friction cam, and wherein the measurement indicates an angle of rotation of the friction cam.

7. The motorized surgical handle assembly apparatus of claim 5, wherein the sensor comprises a binary switch that is actuatable via movement of the friction cam, and wherein the measurement indicates that the friction cam has moved by an amount corresponding to movement of the movable handle by at least the threshold amount.

8. The motorized surgical handle assembly apparatus of claim 5, wherein the friction cam includes a magnet, wherein the sensor comprises a Hall effect sensor, and wherein the measurement indicates a position or an angle of rotation of the magnet of the friction cam.

9. A method of operating a motorized surgical handle assembly apparatus, the method comprising:
receiving a measurement from a sensor of the motorized surgical handle assembly apparatus, wherein the measurement corresponds to at least one of a position or orientation of a movable handle of the motorized surgical handle assembly apparatus;
determining, based on the received measurement, that the movable handle has moved away from a non-movable handle of the motorized surgical handle assembly apparatus by at least a threshold amount; and
operating, based on the determination that the movable handle has moved away from the non-movable handle by at least the threshold amount, a drivetrain of the motorized surgical handle assembly apparatus to retract a drive assembly of the motorized surgical handle assembly apparatus.

10. The method of claim 9, wherein operating the drivetrain includes operating the drivetrain such that the movable handle is moved away from the non-movable handle.

11. The method of claim 9, wherein operating the drivetrain includes operating the drivetrain such that jaws of a reloadable cartridge assembly coupled to the motorized surgical handle assembly apparatus are unclamped.

12. The method of claim 9, wherein receiving the measurement from the sensor includes receiving the measurement from a rotational potentiometer coupled to the movable handle, and wherein the measurement indicates an angle of rotation of the movable handle.

13. The method of claim 9, wherein:
receiving the measurement from the sensor includes receiving the measurement from (a) a rotational potentiometer coupled to a friction cam movably coupled to the non-movable handle or (b) a Hall effect sensor; and
the measurement indicates an angle of rotation of the friction cam or an angle of rotation of a magnet included in the friction cam.

14. The method of claim 9, wherein receiving the measurement from the sensor includes receiving the measurement from a binary switch, and wherein the measurement comprises an indication that a friction cam movably coupled to the non-movable handle has moved by an amount corresponding to movement of the movable handle by at least the threshold amount.

15. A motorized surgical handle assembly apparatus, comprising:
a non-movable handle;
a movable handle movably coupled to the non-movable handle;
a drivetrain operably coupled to the movable handle and coupleable to a reloadable cartridge assembly having jaws;
a sensor configured to obtain a measurement corresponding to at least one of a position or orientation of the movable handle; and
a controller operably coupled to the drivetrain and the sensor, wherein the controller is configured to operate the drivetrain to clamp or unclamp the jaws by an amount corresponding to the measurement.

16. The motorized surgical handle assembly apparatus of claim 15 wherein the controller is configured to operate the drivetrain such that an angle between the jaws changes at a first rate corresponding to a second rate at which the position or orientation of the movable handle changes.

17. The motorized surgical handle assembly apparatus of claim 15 wherein the controller is configured to operate the drivetrain to unclamp the jaws as the movable handle is moved distally, and wherein the amount of unclamping of the jaws increases incrementally as the movable handle is moved distally.

18. The motorized surgical handle assembly apparatus of claim 15 wherein the controller is configured to operate the drivetrain to clamp the jaws as the movable handle is moved proximally, and wherein the amount of clamping of the jaws increases incrementally as the movable handle is moved proximally.

19. The motorized surgical handle assembly apparatus of claim 15 wherein the controller is configured to operate the drivetrain such that an angle between the jaws changes linearly with the position or orientation of the movable handle.

20. The motorized surgical handle assembly apparatus of claim 15 wherein the controller is configured to operate the drivetrain such that an angle between the jaws changes non-linearly with the position or orientation of the movable handle.
